SEQUENCE LISTING (8 sequences)

SEQ ID NO: 1 - protein, 985 aa, Yersinia pseudotuberculosis
MVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAVPTSIIADGLMASTITLELKDTYGDPQAGANVAFDTTLGNMGVITDHNDGTYSAPLTSTTLGVATVTVKVDGAAFSVPSVTVNFTADPIPDAGRSSFTVSTPDILADGTMSSTLSFVPVDKNGHFISGMQGLSFTQNGVPVSISPITEQPDSYTATVVGNTAGDVTITPQVDTLILSTLQKKISLFPVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGTSLVSSLEASRQCQGSDMSAVLESSRATNGTRAPDGTLWGEWGSLTAYSSDWQSGEYWVKKTSTDFETMNMDTGALVQGPAYLAFPLCALAI

SEQ ID NO: 2 - protein, 529 aa, Listeria monocytogenes
MKKIMLVFITLILISLPIAQQTEAKDASAFHKEDLISSMAPPTSPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

SEQ ID NO: 3 - DNA, 1207 bp, Artificial Sequence
  Synthetic Sequence
agtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttggtaccgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 4 - DNA, 1320 bp, Artificial Sequence
  Synthetic Sequence
agtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttggtaccgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgagtaagtcgactcgttggatccccactacagccgatactcaagcttgacgaattcgagtatccaaggtagtggactagtgtgacgctgctgacccctttctttcccttctgcagcaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 5 - DNA, 1270 bp, Artificial Sequence
  Synthetic sequence
ccgtcgcggcaggtgttatacctgcgattagcagaaaaaagaaaacaaattttatctttgtcatggctgaaaatccgatatcatcttgccgtttataaacccatccctggatgtggaatagcatactccggtcatatctgtttagcgaggttttgcactggtgggcagaacctgaaaaaaaggaatacaaatgcaagcggcaactgttttgattaaccgccgcgctctgcgacacaacctgcaacgtctgcgtgaactggcccccgccagtaaactggttgcggtggtgaaagcgaacgcctacggtcacggtctgattgagaccgcgcgaacgctccccgatgctgacgcctttggcgttgcccgtctcgaagaagccctacggctgcgggcggggggtatcacgcgacctattttgttactggaaggtttttttgaagcagacgatttgccgacgatctccgctgaacatctgcataccgcagtccacaatgaagagcagcttgtcgccctcgaaaacgctgaacttaaagagcctgtcaccgtctggatgaagctcgataccggaatgcaccgtttgggcgtattgccggaacaggccgaggcgttttatcagcgtctgagccagtgtaaaaatgtccgccagccggtgaacattgttagtcacttcgcccgtgccgatgaaccgcaaagcggcgcgactgaaaagcagctcgatatcttcaacaccttttgtgaaggtaaaccggggcaacgctcaattgcggcatcaggcggcattttgttatggccgcagtcgcattttgactgggcgcgtccggggatcattctttacggtgtctcgccgctggaagatggcacgacaggggctgattttggctgtcagccagtcatgtctttaacctccagcctgattgccgtgcgtgagcataaagccggagagcctgtcggttatggtggaacctgggtaagcgaacgtgatactcgtcttggcgtagtcgcgatgggctatggcgatggttatccgcgcgccgcgccgtccggtacgccagtgctggtgaacggtcgcgaagtgccgattgtcgggcgagtcgcgatggatatgatctgcgtagacttaggtccacaggcgcaggacaaagccggggacccggtcattttatggggcgaaggtttgcccgtagaacgtatcgctgaaatgacgaaagtaagcgcttacgaacttattacgcgcctgacttcaagagtcgcgatgaaatacgtggattaa

SEQ ID NO: 6 - DNA, 1260 bp, Artificial Sequence
  Synthetic Sequence
agtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctgccaagcttggtaccgccaccatggagatgttccagggactgctgttacttcttcttttgagcatgggaggtacgtgggcttctaaggagccattacgtccacgctgccgtcctattaacgccaccttggccgtagagaaggaaggctgtcccgtctgtattacagtgaacactacaatctgcgctgggtattgtccaactatgacccgtgtcttgcaaggggttcttcccgcctgtaagtcgactcgttggatccccactacagccgatactcaagcttgacgaattcgagtatccaaggtagtggactagtgtgacgctgctgacccctttctttcccttctgcagtaccacaagtagtctgcaattaccgcgatgtccgttttgaaagtatccgtttgccgggatgtccgcgcggagtgaacccggtggtgtcctacgcggttgcactgagctgtcagtgcgctttatgtcgccgcagtaccacggattgcggagggcccaaggatcaccctttgacctgtgatgatccacgtttccaggacagtagtagctcaaaagcacctcctccgagccttccaagcccctcccgtctgccaggcccgtcggatacccccattttaccacagtaa

SEQ ID NO: 7 - DNA, 3213 bp, Artificial Sequence
  Synthetic Sequence
cacagctaacaccacgtcgtccctatctgctgccctaggtctatgagtggttgctggataactttacgggcatgcataaggctcgtataatatattcagggagaccacaacggtttccctctacaaataattttgtttaactttactagttgtgtaggctggagctgcttcgaagttcctatactttctagagaataggaacttcggaataggaactaaggaggatattcatatgatgaattcaggagggtcgacatggtctttcagccgatatccgagttcttactgatacgtaacgccggtatgtccatgtactttaacaaaattatatcgttcaacattatctcaagaattgtcatttgtattttcttgatttgcgggatgttcatggctggcgcctcggagaaatacgacgctaatgcgcctcagcaggtgcaaccctattcagtttcttcgagtgcctttgagaacttgcaccctaataatgaaatggaaagtagtataaatccattctcggcctcagacactgaacgcaacgccgcgataattgaccgcgctaacaaagaacaagaaacggaagccgtcaataagatgatctccacaggggctagattagccgcgagcggacgggcttcagatgttgcgcactctatggttggtgacgcggtgaatcaggagatcaagcaatggttgaatagattcggtactgctcaggtgaacttaaactttgacaaaaacttcagccttaaagagtcttcattagactggttagccccatggtacgactcagcatcctttttattttttagccaattgggcatcagaaataaagactctcgcaacactctgaaccttggagttggaatacgcaccttagagaacggttggctgtatggtttgaatactttctacgataatgacctgactggacacaaccatcgtataggtcttggggcggaagcatggacagactacttacagcttgccgcaaatgggtactttagattaaacggctggcatagtagccgtgatttctctgattataaggagagacctgctacgggaggagatttaagagctaacgcatacttaccagctttaccacagttaggaggcaaactgatgtatgagcagtacactggggagcgggtcgccctttttgggaaggataatctgcagcgcaacccttatgcggtcacggctgggattaactatactcccgtcccgttattgaccgtgggcgtagatcagcgtatgggtaagagttccaagcatgaaacgcaatggaatttgcagatgaactaccgtcttggtgaatccttccagagtcagcttagccccagcgcggtcgctggtactcgcctgttagcagaaagtcggtacaatttagtagatagaaacaacaacatagtcttagagtatcagaaacaacaggttgttaagcttacattgtctcccgccaccataagcgggctgcctggacaagtctatcaagttaacgcccaggtgcaaggtgcgagcgccgttcgggaaatagtgtggagcgatgcggaacttatcgccgcgggaggaacgttaacgcctttatccacaacccagtttaacttagtgttgcccccctacaagagaaccgcgcaggtatcgcgcgtaaccgacgatttaacagctaacttctacagtctttccgcattagccgtggaccaccaaggaaacagaagtaactccttcactcttagcgtcactgttcaacagccacaactgactctgacggctgcggtaattggtgatggggcgccagccaatgggaagactgcaattacggttgagttcaccgtagcggatttcgaggggaagccacttgcagggcaagaagtagtaataacaactaacaacggtgctcttcctaacaaaattacagaaaagacggacgcaaacggtgttgctcgcatcgcattaacgaataccactgatggagtaaccgtggtcactgcagaagttgaggggcaacgccagtcggtggatactcatttcgtcaaggggacgatagcagcagacaaatctacgcttgcggccgtgcctaccagcatcatcgcggacggactgatggcatcgaccattacccttgagttgaaggacacctacggcgatcctcaggcgggtgctaatgtggcattcgatactacactgggcaacatgggagtaataactgaccataacgatggtacttattccgcacccttaacaagtactacgttgggtgtagctaccgtaaccgttaaagtcgatggagccgctttttcggttccgtcggtcaccgtcaatttcacagctgatccgataccagatgcgggccgcagcagcttcaccgtcagtactccggacatattagctgacgggactatgtcttctacgttgtcctttgtacccgtcgataagaacggtcattttattagcgggatgcagggtttgtcatttacgcagaatggcgtccctgtatcaatctcgcctattacggagcaacccgattcctacacagcaacggtagttggaaatacggcaggtgatgtaacaataacaccccaagttgacacactgatcctgtcaaccttgcagaaaaagatctcgttgtttcctgttccgaccttaacgggaatcttagtaaatggtcagaattttgccactgacaaggggttccctaagaccatcttcaaaaatgctacattccagttacagatggacaatgacgtggccaacaacactcaatacgaatggtcctccagcttcactcctaatgtatcggttaatgaccaaggacaggtaaccattacctaccaaacctacagcgaggtcgccgtaactgcgaaatcaaaaaagtttccatcctatagcgtatcttacagattctatccgaacagatggatttacgatggaggcacaagtctggtaagctcgctggaagcatcacggcaatgccaaggtagtgatatgagcgccgtcctggaaagctcgagagctacaaatggaaccagagctccagatgggactttgtggggtgagtggggcagtttgaccgcgtacagttcggattggcagtctggggaatattgggtaaaaaagacttccacagatttcgaaacgatgaatatggataccggggcgcttgtccaaggtcccgcatatttggcgttcccgctttgcgctttagccatctag

SEQ ID NO: 8 - protein, 505 aa, Artificial Sequence
  Synthetic Sequence
MDASAFHKEDLISSMAPPTSPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

(19) United States
(12) Patent Application Publication
(10) Pub. No.: US 2024/0118283 A1
(43) Pub. Date: Apr. 11, 2024

(54) ENGINEERED MICROORGANISMS FOR DETECTION OF DISEASED CELLS

(71) Applicant: Microbial Machines, Inc., Woburn, MA (US)

(72) Inventors: Jeffrey Wagner, Boston, MA (US); Fred H. Mermelstein, West Newton, MA (US); Carl D. Novina, Newton, MA (US); Robert Distel, Framingham, MA (US); Steven Neier, Waltham, MA (US); Barry Polisky, Boulder, CO (US)

(21) Appl. No.: 18/533,644

(22) Filed: Dec. 8, 2023

Related U.S. Application Data
(63) Continuation of application No. PCT/US2022/032789, filed on Jun. 9, 2022.
(60) Provisional application No. 63/208,888, filed on Jun. 9, 2021.

(51) Int. Cl. G01N 33/574; C12N 1/20

(52) U.S. Cl. CPC G01N 33/57446; C12N 1/20; C12R 2001/19

(57) ABSTRACT

The present invention relates to, inter alia, engineered microorganisms expressing (i) a surface protein, which specifically interacts cell membrane receptors that are specifically exposed to the luminal side of epithelial cells of diseased gastrointestinal tissue, and (ii) a secretable biomarker. The engineered bacteria of the present technology are useful for detecting diseased gastrointestinal tissue.

### CROSS-REFERENCE TO RELATED APPLICATION(S)

The present application is a continuation of International Application No. PCT/US2022/032789, filed Jun. 9, 2022, which claims the benefit of priority to U.S. Provisional Patent Application No. 63/208,888, filed Jun. 9, 2021, each of which is incorporated by reference herein in its entirety.

### TECHNICAL FIELD

The present invention relates, inter alia, to engineered microorganisms and uses thereof.

### BACKGROUND

The gastrointestinal tract (GI tract) takes in food, digests it to extract and absorb energy and nutrients, and expels the remaining waste as feces. Gastrointestinal diseases (GI diseases) are the diseases involving the organs that form the gastrointestinal tract, which include the mouth, esophagus, stomach and small intestine, large intestine and rectum. GI diseases include Barrett's esophagus, inflammatory bowel disease (IBD), irritable bowel syndrome (IBS), Crohn's disease, ulcerative colitis, and precancerous syndromes, and cancer.

The diagnosis of GI diseases starts with symptoms and medical history. Techniques like endoscopy, colonoscopy and computed tomography (CT) scan aid diagnosis by facilitating viewing of the lumen of the GI tract. For example, focal, irregular and asymmetrical gastrointestinal wall thickening on CT scan suggests a malignancy. Segmental or diffuse gastrointestinal wall thickening can indicate an ischemic, inflammatory or infectious disease. However, endoscopy, colonoscopy, etc. are invasive procedures and uncomfortable for the patient, in particular for patients that are at high risk for cancers of the GI tract. A less invasive monitoring technique is desirable.

### SUMMARY

Accordingly, in various aspects, the present invention provides compositions and methods that are useful for detecting diseased epithelial tissue, such as gastrointestinal (GI) tissue or epithelium of ducts that are connected with gastrointestinal tissue (e.g., epithelial tissue lining the bile duct, pancreatic duct, and common bile duct). An aspect of the present invention relates to a method for detecting diseased epithelial tissue, such as gastrointestinal (GI) tissue and epithelium of ducts that are connected with gastrointestinal tissue (e.g., epithelial tissue lining the bile duct, pancreatic duct, and common bile duct). In various embodiments, the methods allow for the presence of diseased tissue to be detected by the presence of a biomarker in a body fluid or excreted sample, which can avoid the use of more invasive techniques such as a colonoscopy. These embodiments are particularly advantageous for individuals with a genetic predisposition to develop cancerous lesions of the gastrointestinal tract, to aid disease monitoring. In various embodiments, the methods comprise administering to the gastrointestinal tract of a subject in need thereof, a genetically engineered microorganism that directs expression of a secretable biomarker specifically in diseased cells. In some embodiments, the secretable biomarker is secreted by the diseased cells. In some embodiments, the secretable biomarker is excreted in body fluids such as urine and saliva. Accordingly, the secretable biomarker may be detected or measured from a sample of blood, serum, plasma, saliva or urine. Therefore, the method further involves obtaining a biological sample from the subject; and measuring the secretable biomarker in the biological sample to thereby detect the presence of diseased epithelial cells.

In various embodiments, the genetically engineered microorganism specifically interacts with diseased epithelial cells through an expressed surface protein that specifically interacts with one or more cell membrane receptor(s) that are specifically present on diseased gastrointestinal epithelial cells (i.e., as compared to non-diseased gastrointestinal epithelial cells). For example, the cell membrane receptor may not be exposed to the luminal side of epithelial cells of normal gastrointestinal tissue, but is exposed to the luminal side of diseased epithelial cells of gastrointestinal tissue in the subject suffering from a disease. The surface protein thereby promotes binding and invasion of the microorganism in the diseased epithelial cells. In various embodiments, the microorganism comprises a gene encoding the secretable biomarker operably linked to a promoter. Thereby, in various embodiments, the microorganism delivers a nucleic acid (e.g. a DNA or an mRNA molecule) or protein to diseased epithelial cells. In various embodiments, the diseased epithelial cells secrete the secretable biomarker, and thereby allowing convenient detection of the diseased epithelial cells by simple evaluation of biological fluid samples. In various embodiments, the secretable biomarker is a secreted protein. In some embodiments, the secretable biomarker is excreted in a biological fluid. In some embodiments, the biological fluid is selected from mucus, saliva and/or urine. In some embodiments, the secretable biomarker is detected in a blood or feces sample.

In various embodiments, the detection of diseased epithelial tissue (e.g., of the gastrointestinal tract) comprises measuring the secretable biomarker in the biological sample obtained from the subject. In any of the embodiments disclosed herein, the detection may be performed using an enzymatic test or immunoassay.

In various embodiments, the genetically engineered microorganism is administered via oral or rectal route. In various embodiments, optionally a colon cleansing agent may be administered prior to and/or after the administration of the microorganism. In various embodiments, the method disclosed herein detects diseased gastrointestinal (GI) tissue selected from a precancerous lesion, cancer, or a lesion caused by Lynch Syndrome, ulcerative colitis, Crohn's disease, Barrett's esophagus, irritable bowel syndrome and/or irritable bowel disease. In various embodiments, the genetically engineered microorganism is administered via oral or rectal route. In various embodiments, optionally a colon cleansing agent may be administered prior to and/or after the administration of the microorganism.

In various embodiments, the subject is predisposed to develop polyps and/or cancer (without limitation, e.g. colorectal cancer, pancreatic ductal adenocarcinoma or cholangiocarcinoma). In some embodiments, the subject suffers from a condition selected from Lynch Syndrome, hereditary non-polyposis colon cancer (HNPCC), familial adenomatous polyposis (FAP), Gardner's Syndrome, Turcot's Syndrome, MUTYH-associated polyposis, Peutz-Jeghers syndrome, and juvenile polyposis syndrome and colitis-associated colorectal cancer (CACC).

An aspect of the present invention relates to a genetically engineered microorganism. The microorganism comprises a gene encoding a surface protein that specifically interacts with diseased epithelial cells via one or more cell membrane receptor(s) that are exposed to the luminal side of diseased epithelial cells of gastrointestinal tissue. The one or more cell membrane receptor(s) are not expressed on the luminal side of epithelial cells of normal gastrointestinal tissue, thus conferring specificity for diseased or abnormal cells of gastrointestinal tissue on the microorganism. The surface protein specifically promotes the invasion of epithelial cells of diseased gastrointestinal tissue.

In various embodiments, the microorganism is non-pathogenic. In various embodiments, the microorganism harbors at least one auxotrophic mutation. In various embodiments, the at least one auxotrophic mutation allows for selection and containment of the microorganism, and also facilitates lysis of the microorganism inside the diseased mammalian cell upon invasion. Exemplary auxotrophic mutations include deletion, inactivation, or reduced activity or expression of one or more genes involved in synthesis of metabolites required for cell wall synthesis. Exemplary metabolites involved in cell wall synthesis include D-alanine and diaminopimelic acid. In various embodiments, the microorganism further comprises a gene encoding a lysin, which causes lysis of a phagosome.

In various embodiments, the microorganism comprises a gene encoding the secretable biomarker (secretable by mammalian cells) operably linked to a promoter. In some embodiments, the secretable biomarker is expressed from a mammalian promoter. In some embodiments, the mammalian promoter is active or specific for epithelial expression or GI tract epithelial cell-specific expression and/or specific expression in the epithelium of ducts that are connected with gastrointestinal tissue (e.g., epithelial tissue lining the bile duct, pancreatic duct, and common bile duct). In these embodiments, the microorganism delivers a DNA molecule (e.g. a plasmid) to diseased epithelial cells. The plasmid can be a single copy plasmid, or in some embodiments a multi-copy plasmid (e.g., a high copy number plasmid). In these embodiments, the DNA molecule optionally comprises at least one binding site for a DNA binding protein. In these embodiments, the DNA molecule optionally comprises at least one binding site for an exogenous DNA binding protein. In some embodiments, the DNA binding protein comprises one or more nuclear localization signal(s) (NLS), thus allowing nuclear translocation of the DNA molecule (e.g. a plasmid) in the diseased epithelial cells. In these embodiments, the diseased epithelial cells express the secretable biomarker from the DNA molecule (e.g. a plasmid) delivered by the microorganism, thereby allowing their detection.

In alternative embodiments, the secretable biomarker is expressed from a microbial promoter, including for example, a T7 or T3 promoter. In such embodiments, the microorganism will further comprise and express a gene for a T7 or T3 RNA polymerase. Other suitable microbial promoters are further described herein and known in the art. In these embodiments, the gene encoding the secretable biomarker is constructed for translation in the mammalian cell. In some embodiments, the gene encoding the secretable biomarker comprises an internal ribosome entry site. In these embodiments, the microorganism delivers an mRNA molecule to diseased epithelial cells, which is translated in the mammalian cell. In these embodiments, the diseased epithelial cells express the secretable biomarker from the mRNA molecule delivered by the microorganism, thereby allowing for convenient detection of the diseased epithelial cells.

In alternative embodiments, the promoter is a microbial promoter, and the expressed mRNA is translated in the bacterial cell. In these embodiments, the one or more gene(s) encoding at least one detection marker optionally further comprises a protein that becomes fluorescent upon contact with a metabolite found only in the mammalian cytoplasm. In these embodiments, the microorganism produces and delivers the protein molecules to diseased epithelial cells. In these embodiments, the diseased epithelial cells do not produce the protein, but instead become fluorescent when the protein produced by the microorganism encounters the metabolite found only in the mammalian cytoplasm.

In various embodiments, the secretable biomarker is a secreted protein selected from an enzyme, a peptide hormone, or a peptide or protein antigen. Examples include alkaline phosphatase, a human chorionic gonadotropin, a human carcinoembryonic antigen (CEA), colon cancer secreted protein 2, Cathepsin B, a Gaussia luciferase (Gluc), a Metridia luciferase (MLuc), a subunit thereof, a fragment thereof, and a combination of any two or more thereof.

In various embodiments, the microorganism is selected from Lactobacillus, Bifidobacterium, Saccharomyces, Enterococcus, Streptococcus, Pediococcus, Leuconostoc, Bacillus, Escherichia coli. In some embodiments, the microorganism is Escherichia coli. An exemplary E. coli strain is E. coli Nissle 1917 or a derivative thereof.

In various embodiments, the gene encoding the secretable biomarker may be inserted on a natural endogenous plasmid from Escherichia coli Nissle 1917 (i.e., pMUT1, pMUT2, and/or a derivative thereof). In some embodiments, the plasmid comprises a selection mechanism. In some embodiments, the selection mechanism may not require an antibiotic for plasmid maintenance. Accordingly, in some embodiments, the selection mechanism is selected from an antibiotic resistance marker, a toxin-antitoxin system, a marker causing complementation of a mutation in an essential gene, a cis acting genetic element and a combination of any two or more thereof. In some embodiments, the plasmid carrying the gene encoding the secretable marker complements an auxotrophic mutation, such as an auxotrophic mutation that deletes or inactivates a gene involved in synthesis of metabolites required for cell wall synthesis.

An aspect of the present invention relates to a method of diagnosis of a disease in a subject, the method comprising: (i) administering to target epithelial cells of interest (such as epithelial cells of the gastrointestinal tract) of the subject the genetically engineered microorganism disclosed herein, and (ii) detecting the expression of the secretable biomarker to thereby detect the presence of diseased epithelial cells.

An aspect of the present invention relates to a method of diagnosis and/or treatment of a disease in a subject, the method comprising: (i) administering to target epithelial cells (such as epithelial cells of the gastrointestinal tract) of the subject a genetically engineered microorganism of any of the embodiments disclosed herein; and (ii) obtaining a biological sample from the subject; and (iii) measuring the secretable biomarker in the biological sample to thereby detect the presence of diseased epithelial cells. Where diseased epithelial cells are detected, the method may further comprise administering a treatment to the subject, and/or performing a colonoscopy to locate, evaluate, and/or remove the diseased tissue.

An aspect of the present invention relates to a method of selecting a subject suffering from or suspected to be suffering from a disease for a treatment, the method comprising: (i) administering to target epithelial cells (such as epithelial cells of the gastrointestinal tract) of the subject a genetically engineered microorganism of any of the embodiments disclosed herein; (ii) obtaining a biological sample from the subject; and (iii) measuring the secretable biomarker in the biological sample to thereby detect the presence of diseased epithelial cells; and (iv) selecting the subject for treatment if expression of the secretable biomarker is observed. In some embodiments, the treatment comprises a colonoscopy optionally with polyp or lesion resection. In some embodiments, the treatment alternatively or in addition comprises chemotherapy, radiation therapy, or immunotherapy.

An aspect of the present invention relates to a method for treating a cancer in a patient, comprising: (i) administering to target epithelial cells (such as epithelial cells of the gastrointestinal tract) of the subject a genetically engineered microorganism of any of the embodiments disclosed herein; (ii) detecting the expression of the secretable biomarker to thereby detect the presence of diseased epithelial cells; and (iii) administering a treatment if the expression of the secretable biomarker is observed. In various aspects and embodiments, the treatment is surgery or administration of a therapeutic agent selected from the group consisting of a chemotherapeutic agent, a cytotoxic agent, an immune checkpoint inhibitor, an immunosuppressive agent, a sulfa drug, a corticosteroid, an antibiotic and a combination of any two or more thereof.

Other aspects of the present invention provide a genetically engineered microorganism of any of the embodiments disclosed herein for use in the method of the above aspects.

Any aspect or embodiment disclosed herein can be combined with any other aspect or embodiment as disclosed herein.

### BRIEF DESCRIPTION OF THE DRAWINGS

FIG. 1 shows a schematic representation of diseased cells of GI tract epithelium. Basolateral and lumen sides are shown. The middle cell is a diseased cell, which exhibits mislocalized receptors displayed on the lumen side. Other diseased cells may have mislocalized and/or novel mammalian membrane receptors. The novel receptors may be the receptors that are normally not found in the cells or the receptors formed by translocations and other genomic rearrangement.

FIG. 2 shows a schematic representation of E. coli Nissle 1917 (EcN) strain. This strain is an exemplary strain useful for producing the genetically engineered bacterium. Chromosome and naturally occurring plasmids pMUT1 (GenBank Accession No. MW240712) and/or the plasmid pMUT2 (GenBank Accession No. CP023342) are represented by circles of different sizes.

FIG. 3 shows a schematic representation of an embodiment of the base strain of the genetically engineered E. coli Nissle 1917 (EcN) strain harboring one or more auxotrophic mutation(s) (shown by X). Exemplary auxotrophic mutations include dapAΔ, alrΔ, and dadXΔ. Such mutations prevent the bacterial cell from propagating in the body or the environment, and thereby aid in containment of the genetically engineered bacterium.

FIG. 4A and FIG. 4B shows growth requirements and growth characteristics of a genetically engineered E. coli Nissle 1917 (EcN) strain harboring dapAΔ, alrΔ, dadXΔ auxotrophic mutations. Double deletion of alr and dadX, the genes that encode alanine racemase, results in a D-alanine auxotrophy. Deletion of dapA results in auxotrophy for diaminopimelic acid. The graph in FIG. 4A demonstrates that the strain grows only when both D-alanine and diaminopimelic acid are added to growth media. The graph in FIG. 4B demonstrates that that when D-alanine and diaminopimelic acid are added to the media, the strain grows similarly to the wild type strain.

FIG. 5 shows a schematic representation of an embodiment of the genetically engineered bacterium E. coli Nissle 1917 (EcN) strain having genes encoding a surface protein and listeriolysin O integrated in the genome. Exemplary surface proteins are invasin (SEQ ID NO: 1) and a nanobody/receptor binding peptide expressed on a bacterial scaffold. Listeriolysin O (SEQ ID NO: 2) is expressed to allow escape from the endosome.

FIG. 6 shows a schematic representation of an embodiment of the genetically engineered E. coli Nissle 1917 (EcN) derivative harboring one or more auxotrophic mutation(s) (shown by X), further having genes encoding surface protein and listeriolysin O integrated in the genome. This strain does not contain plasmid pMUT1.

FIG. 7A and FIG. 7B shows results of curing the cryptic plasmids from E. coli Nissle 1917 (EcN). FIG. 7A shows an agarose gel showing the sequential curing of pMut1 and then pMut 2. Wild type E. coli Nissle 1917 (EcN) was transformed with a curing plasmid and passaged in the presence of 5 mg/ml ampicillin. Plasmid preparations from wild type E. coli Nissle 1917 (EcN) (lane A), E. coli Nissle 1917 (EcN) cured of pMUT1 (lane B), and E. coli Nissle 1917 (EcN) cured of pMUT1 and pMUT2 (lane C) FIG. 7B shows the results of quantitative PCR confirming the curing of pMUT1 and pMUT2 in the final strain. qPCR was carried out with primers specific to rpoA (chromosomal marker), pMUT1 and pMUT2 and show are the inverse of the cycle number where the amplification passed a set threshold (Cq⁻¹). Data labels are as in FIG. 7A.

FIG. 8 shows a schematic representation of a non-limiting embodiment of the genetically engineered bacterium of present disclosure. This strain is an E. coli Nissle 1917 (EcN) derivative harboring one or more auxotrophic mutation(s) (shown by X), further having genes encoding surface protein and listeriolysin O integrated in the genome. This strain does not contain the plasmid pMUT1, but contains the plasmid pSRX, a pMUT1-based derivative, which is selected using complementation of at least one of the auxotrophic mutations as the selection mechanism (e.g., complementation of alr and dadX by plasmid borne alr gene). Plasmid pSRX also carries a detection marker, which is exemplified herein by GFP.

FIG. 9A to FIG. 9D show, without being bound by theory, a schematic representation of the method for detecting diseased gastrointestinal (GI) tissue. FIG. 9A shows specific binding of the genetically engineered bacterium to diseased epithelial cells (represented by the middle cell), which show a mislocalized receptor that is displayed on the lumen side of GI tract. Such binding leads to the internalization in the diseased epithelial cells (represented by the middle cell) of the genetically engineered bacterium. FIG. 9B shows bacterial lysis due to attenuation mutation, and lysis of phagosome through the action of LLO. FIG. 9C shows nuclear localization of the plasmid harboring the secretable biomarker upon lysis of the genetically engineered bacterium. FIG. 9D shows expression of the secretable biomarker by the diseased epithelial cells (represented by the middle cell) of GI tract.

FIG. 10 shows the expression of the detection marker (GFP) expressed by bacterial cells after invading SW480 colorectal cancer cells in vitro. A bacterial strain containing mNeonGreen (a green fluorescent protein) without (top panels) or with (bottom panels) an invasin gene after coincubating with SW480 (colorectal cancer derived cell line) for one hour, followed by washing away of extracellular bacteria. The SW480 cells were visualized by fluorescence microscopy (left panels), removed from the plate, and then analyzed by flow cytometry (right panels) to identify the portion of the SW480 cells that were successfully invaded by the bacterial strain.

FIG. 11A to FIG. 11C show a schematic representation of the non-invasive test for diseased cells disclosed here. FIG. 11A illustrates, without being bound by theory, the mechanism of delivery of DNA payload. FIG. 11B (left panel) shows a target cancer cell with the DNA payload delivered by the genetically engineered bacterium (without limitation, e.g., E. coli Nissle 1917). The middle panel illustrates the expression and secretion of the detection marker encoded by the DNA payload. The right panel represents a blood vessel with the detection marker. In some embodiments, the detection marker is secreted in the urine. FIG. 11C illustrates that the detection marker may be detected with a simple blood or urine test.

FIG. 12A to FIG. 12D demonstrate the in vitro detection of diseased cells using a Gaussia luciferase (Glue) detection marker. FIG. 12A shows the organization of a plasmid harboring a Gluc gene under control of a mammalian promoter (SEQ ID NO: 3). The plasmid has genes encoding listeriolysin O (lysin), and a selection marker e.g. Amp^{R} or alr. FIG. 12B shows the expression of cancer Gluc by cells contacted with E. coli Nissle 1917-based engineered bacteria of FIG. 12A. FIG. 12C shows the organization of a plasmid harboring a Gluc gene having an intron under control of a mammalian promoter (SEQ ID NO: 4). The plasmid has genes encoding listeriolysin O (lysin), and a selection marker e.g. Amp^{R} or alr. FIG. 12D shows the expression of Gluc by cancer cells contacted with E. coli Nissle 1917-based engineered bacteria of FIG. 12C.

FIG. 13A to FIG. 13B demonstrate the in vitro detection of diseased cells using the 0-chorionic gonadotropin (hCG) detection marker. Detection of hCG produced by a cancer cell line upon delivery of DNA payload by E. coli Nissle 1917-based engineered bacteria using ELISA-based test (FIG. 13A) and a pregnancy test (FIG. 13B) are shown.

### DETAILED DESCRIPTION

Current diagnosis of abnormally growing cells in the gastrointestinal tract is based upon invasive colonoscopies that are not comfortable for the patient and not always successful in detection of diseased cells. The ability to visualize and remove abnormal cells and diseased tissue varies depending on the skills of the surgeon and prominence of the polyps or tumors. Certain abnormally growing cells are flat or small in number and therefore, not visualized and removed by even skilled surgeons. Further, for patients that have a condition that involves increased risk of colorectal cancer, frequent colonoscopies are a substantial burden. The present disclosure provides engineered bacterial cells that are to be administered for the purpose of detecting the abnormal cells in a subject by routine analysis of body fluid samples. The engineered bacterial cells have been genetically altered to invade abnormal cells and deliver a nucleic acid encoding a secretable biomarker, enabling the abnormal cells to express and secrete the secretable biomarker. In some embodiments, the secretable biomarker is excreted in a biological sample such as urine, mucus, saliva, and feces. In some embodiments, the detection maker is secreted in blood, and thus may be detected in a sample such as blood, serum or plasma. In various embodiments, the secretable biomarker is detected using an enzymatic assay or immunoassay, optionally using modalities such as agglutination, luminescence or fluorescence, dipstick assay, and lateral flow immunoassay.

In some embodiments, the secretable biomarker is optimized for distribution in body fluids (e.g. decreased or increased excretion, and increased stability). In some embodiments, the secretable biomarker is optimized for distribution in the blood. In some embodiments, the secretable biomarker is optimized for distribution in the urine. Accordingly, in some embodiments, the secretable biomarker is optimized for increased excretion (e.g. in urine or feces). In alternative embodiments, the secretable biomarker is optimized for decreased excretion, leading to accumulation of the secretable biomarker in the blood. In some embodiments, the secretable biomarker is optimized for increased stability (e.g. improved protein folding and/or stability against proteolysis) leading to increased accumulation. In exemplary embodiment, the secretable biomarker is optimized for increased stability and decreased excretion, leading to accumulation in the blood. In another exemplary embodiment, the secretable biomarker has increased stability and increased excretion, leading to accumulation in the urine.

Accordingly, in various aspects, the present invention provides compositions and methods that are useful for detecting diseased epithelial cells, such as epithelial cells of gastrointestinal (GI) tissue or diseased epithelium of ducts that are connected with gastrointestinal tissue (e.g., epithelial tissue lining the bile duct, pancreatic duct, and common bile duct). An aspect of the present invention relates to a method for detecting diseased epithelial cells, such as epithelial cells of gastrointestinal (GI) tissue or epithelial cells of ducts that are connected with gastrointestinal tissue (e.g., epithelial tissue lining the bile duct, pancreatic duct, and common bile duct) comprising (i) administering to the target epithelial cells (e.g., of the gastrointestinal tract) of a subject in need thereof, a genetically engineered microorganism, (ii) obtaining a biological sample from the subject; and (iii) measuring the secretable biomarker in the biological sample to thereby detect the presence of diseased epithelial cells. In some embodiments, the genetically engineered microorganism is non-pathogenic, auxotrophic, and comprises an exogenous gene encoding a surface protein that specifically interacts with one or more cell membrane receptor(s). In some embodiments, the cell membrane receptor is not exposed to the luminal side of epithelial cells of normal gastrointestinal tissue, but is exposed to the luminal side of diseased epithelial cells (e.g., of gastrointestinal tissue) in the subject suffering from a disease. The surface protein promotes binding and invasion of the microorganism in the diseased epithelial cells. The microorganism also comprises a gene encoding the secretable biomarker operably linked to a promoter to drive mammalian or bacterial expression. In some embodiments, the promoter may be a mammalian promoter. In some embodiments, the mammalian promoter directs epithelial-specific expression or GI tract epithelial cell-specific expression and/or specific expression in the epithelium of ducts that are connected with gastrointestinal tissue (e.g., epithelial tissue lining the bile duct, pancreatic duct, and common bile duct). The genetically engineered microorganism may be administered via oral or rectal route. In this aspect, a colon cleansing agent may optionally be administered prior to and/or after the administration of the microorganism. In any of the embodiments disclosed herein, a biological sample is obtained from the subject; and (iii) the secretable biomarker is measured in the biological sample to thereby detect the presence of diseased epithelial cells. In any of the embodiments disclosed herein, the biological sample may be blood, plasma, serum, mucus, urine, feces, saliva, or a combination of any two or more thereof. In some embodiments, the secretable biomarker is measured without the use of a clinical laboratory instrument selected from a colonoscope, an endoscope, a radiography instrument (e.g. an X-ray machine), a computed tomography (CT) scanner, a magnetic resonance imaging (MRI) machine, a blood gas analyzer, and an urine chemistry analyzer. In any of the embodiments disclosed herein, the secretable biomarker is measured by agglutination, amperometry, atomic absorption spectrometry, atomic emission spectrometry, circular dichroism (CD), chemiluminescence, colorimetry, dipstick assay, lateral flow immunoassay, fluorimetry, electrophoretic assay, enzymatic assay, enzyme linked immunosorbant assay (ELISA), gas chromatography, gravimetry, high pressure liquid chromatography (HPLC), immunoassay, immunofluorometric enzyme assay, mass spectrometry, nuclear magnetic resonance, radioimmunoassay, spectrometry, titrimetry, western blotting, or a combination of any two or more thereof.

The gastrointestinal wall surrounding the lumen of the gastrointestinal tract is made up of four concentric layers called mucosa, submucosa, muscular layer, and serosa (if the tissue is intraperitoneal)/adventitia (if the tissue is retroperitoneal), arranged from the lumen outwards. The characteristics of mucosa depends on the organ. For example, the stomach mucosal epithelium is simple columnar, and is organized into gastric pits and glands to deal with secretion. The small intestinal mucosa, which is made of glandular epithelium intermixed with secretory cells (e.g. goblet cells and Paneth cells), immune cells (e.g. dendritic cells and M cells of the gut-associated lymphoid tissue (GALT)), arranged into villi, creating a brush border and increasing the area for absorption.

The epithelial cells of gastrointestinal tract form a polarized continuous layer. The epithelial cells are connected by tight and adherens junctions, creating a barrier at the apical surface, which controls the selective diffusion of solutes, ions and proteins between the apical and basal tissue compartments. The apical surface of the cells faces the GI tract lumen, and the basolateral surface sits adjacent to an internal-facing basement membrane. The basement membrane is an extracellular matrix (ECM) that comprises laminins, collagen IV, proteoglycans and nidogen. The epithelial cells interact with the ECM through integrins and the transmembrane proteoglycan dystroglycan, which are integral membrane proteins that bind to ECM components as well as intracellular proteins. β1 integrins, which are widely expressed in the epithelial cells, have a central role in establishing their polarity. For example, the binding of integrin to ECM components activates signaling by the integrins, which influences the organization of cytoskeleton, which contributes to cellular polarity.

Disruption of the polarity and barrier function causes diseases. For example, following inactivation of tumor suppressor APC, tissue polarity is lost very early during cancer progression. See, e.g. Fatehullah et al., Philos Trans R Soc Lond B Biol Sci. 368(1629): 20130014 (2013). Thus, the mislocalization of integrins at the opposing basal surface domain correlated with loss of epithelial architecture and cancer development. Krishnan et al., Mol Biol Cell 24(6):818-31 (2013). Similarly, pathogens such as enteropathogenic Escherichia coli and Y. pseudotuberculosis disrupt cell polarity and enable the apical migration of basolateral membrane proteins. Muza-Moons et al., Infect Immun. 71(12): 7069-7078 (2003); McCormick et al., Infect Immun. 65(4):1414-21 (1997). Moreover, diseases such as Crohn's disease, untreated celiac disease, irritable bowel syndrome, and irritable bowel disease feature disruption of the barrier function. Marchiando et al., Annu Rev Pathol 5: 119-144 (2010). Therefore, the detection of mislocalized and/or aberrantly expressed cell surface molecules has great diagnostic value.

A bile duct is a long tube-like structures that carry bile. Small bile ducts are visible in portal triads of liver lobule, which also contain a small hepatic artery branch, a portal vein branch. The small bile ducts fuse to form larger bile ducts. The larger bile ducts in the hepatic triads coalesce to intrahepatic bile ducts that become the right and left hepatic ducts that fuse at the undersurface of the liver to become the common bile duct. About halfway down the common bile duct, the cystic duct (carrying bile to and from the gallbladder) branches off to the gallbladder. The common bile duct opens into the intestine. The intrahepatic ducts, cystic duct, and the common bile duct are lined by a tall columnar epithelium.

The gallbladder stores bile excreted from the liver. The columnar mucosa is arranged in folds over the lamina propria, allowing expansion. Beneath the lamina propria is a muscularis, and surrounding the gallbladder is a connective tissue layer and serosa. The gallbladder mucosa transports out sodium in the bile, passively followed by chloride and water. Thus, bile excreted by the liver and stored in the gallbladder becomes more concentrated. The muscularis of the gallbladder, contracts under the influence of the hormone cholecystokinin excreted by enteroendocrine cells of the small intestine.

The pancreatic duct, or duct of Wirsung (also, known as the major pancreatic duct), is a duct joining the pancreas to the common bile duct. The pancreatic duct joins the common bile duct just prior to the ampulla of Vater, after which both ducts perforate the medial side of the second portion of the duodenum at the major duodenal papilla. There are many rare anatomical variants as well. Pancreatic ducts are lined by columnar cells with luminal microvilli and glycocalyx and small apical cytoplasmic mucin droplets. In large pancreatic ducts, many epithelial cells also have cilia, which function to aid the downstream movement of exocrine secretions.

Accordingly, in various aspects, the present invention provides compositions and methods that are useful for detecting diseased epithelial tissue, such as diseased epithelial tissue of GI tissue or epithelial cells of ducts that are connected with gastrointestinal tissue (e.g., epithelial tissue lining the bile duct, pancreatic duct, and common bile duct). An aspect of the present invention relates to a method for detecting diseased GI tissue comprising (i) administering to the gastrointestinal tract of a subject in need thereof, a genetically engineered microorganism engineered to direct expression of a detectable marker specifically in diseased epithelial cells of the GI tract or epithelium of ducts that are connected with gastrointestinal tissue (e.g., epithelial tissue lining the bile duct, pancreatic duct, and common bile duct), and (ii) detecting the expression of a secretable biomarker to thereby detect the presence of diseased epithelial cells. In various embodiments, the methods comprise administering to the GI tract of a subject in need thereof, a genetically engineered microorganism that directs expression of a secretable biomarker specifically in diseased cells. The method further involves detecting the expression of the secretable biomarker to thereby detect the presence of diseased epithelial cells. In some embodiments, the genetically engineered microorganism is non-pathogenic, auxotrophic, and comprises an exogenous gene encoding a surface protein that specifically interacts with one or more cell membrane receptor(s). The cell membrane receptor is not exposed to the luminal side of epithelial cells of normal epithelial or gastrointestinal tissue, but is exposed to the luminal side of diseased epithelial cells of gastrointestinal tissue in the subject suffering from a disease. Thus, the expression and/or localization of the one or more cell membrane receptor(s) confers the specificity for diseased or abnormal cells of epithelial or gastrointestinal tissue on the microorganism. The surface protein thus promotes binding and invasion of the microorganism in the diseased epithelial cells. The surface protein promotes binding and invasion of the microorganism in the diseased epithelial cells. The microorganism also comprises a gene encoding the secretable biomarker operably linked to a promoter (e.g., a mammalian or bacterial promoter). Thereby, in various embodiments, the microorganism delivers a nucleic acid (e.g., a DNA or an mRNA molecule) to diseased epithelial cells. In various embodiments, the diseased epithelial cells express the secretable biomarker, and thereby allowing their detection. In some embodiments, the promoter may be a mammalian promoter. In some embodiments, the mammalian promoter directs GI tract epithelial cell-specific expression and/or specific expression in epithelium of ducts that are connected with gastrointestinal tissue (e.g., epithelial tissue lining the bile duct, pancreatic duct, and common bile duct).

The diseases that may be diagnosed using the genetically engineered microorganisms, and/or using the methods disclosed herein include precancerous lesions, GI tract cancers, ulcerative colitis, Crohn's disease, Barrett's esophagus, irritable bowel syndrome and irritable bowel disease. GI tract cancers and precancerous syndromes include squamous cell carcinoma of the anus, colorectal cancer (CRC, including colorectal adenocarcinoma, familial adenomatous polyposis, hereditary nonpolyposis colorectal cancer), colorectal polyposis (including Peutz-Jeghers syndrome, juvenile polyposis syndrome, MUTYH-associated polyposis, familial adenomatous polyposis/Gardner's syndrome, and Cronkhite-Canada syndrome), carcinoid, pseudomyxoma peritonei, duodenal adenocarcinoma, distal bile duct carcinomas, pancreatic ductal adenocarcinomas, gastric carcinoma, signet ring cell carcinoma (SRCC), gastric lymphoma (MALT lymphoma), linitis plastic (Brinton's disease), and squamous cell carcinoma of esophagus and adenocarcinoma. The diseased epithelial cells from subjects suffering from one or more of these indications may be detected using the genetically engineered microorganisms of the present disclosure. The genetically engineered microorganisms specifically bind to diseased epithelial cells by specifically interacting with one or more cell membrane receptor(s) that are exposed to the luminal side of diseased epithelial cells of gastrointestinal tissue. The genetically engineered microorganisms do not bind to normal (non-diseased) epithelial cells because the one or more cell membrane receptor(s) or abnormally expressed proteins that occurs in abnormal or diseased cells are not exposed to the luminal side of the normal epithelial cells of gastrointestinal or ductal tissue. In some embodiments, the genetically engineered microorganism delivers one or more nucleic acid(s) encoding secretable biomarker to the diseased epithelial cells (target cells). In these embodiments, the diseased epithelial cells (target cells) express the secretable biomarker, allowing their detection in biological fluid samples. For example, the diseased epithelial cells (target cells) can be identified based on detection of the secretable biomarker in a biological sample such as blood, feces, urine or saliva. Confirmation or evaluation of the diseased epithelial cells may be subsequently carried out using a suitable technique such as colonoscopy, endoscopy, magnetic resonance imaging, CT scan, PET scan, SPECT scan, etc.

Colorectal cancer (CRC) is a common and often lethal tumor. Colorectal adenoma is the most frequent precancerous lesion. Other potentially premalignant conditions include chronic inflammatory bowel diseases and hereditary syndromes, such as familial adenomatous polyposis, Peutz-Jeghers syndrome and juvenile polyposis. These conditions can involve different sites of the gastrointestinal tract. In all such cases, disease recognition at an early stage is essential to devise suitable preventive cancer strategies.

Colorectal adenoma is an asymptomatic lesion often found incidentally during colonoscopy performed for unrelated symptoms or for CRC screening. About 25% men and 15% women who undergo colonoscopic screening have one or more adenomas. Up to 40% of people over the age of 60 harbor colorectal adenomatous polyps as shown in the colonoscopy examinations, although not all colonic polyps are adenomas and more than 90% of adenomas do not progress to cancer.

Lynch syndrome, also known as hereditary non-polyposis colon cancer (HNPCC), accounts for 2-4% of all CRC cases. Individuals with HNPCC have about 75% lifetime risk of developing CRC, and are predisposed to several types of cancer. Colon cancers and polyps arise in Lynch syndrome patients at a younger age than in the general population with sporadic neoplasias, and the tumors develop at a more proximal location. These cancers are often poorly differentiated and mucinous. Muir-Torre syndrome is a variant of Lynch syndrome that presents additional predisposition to certain skin tumors.

Familial adenomatous polyposis (FAP), having a prevalence of 1 in 10,000 individuals, is the second most common genetic syndrome predisposing to CRC. The lifetime risk of developing CRC for individuals suffering from FAP without prophylactic colectomy approaches 100%. The characteristic features of FAP include the development of hundreds to thousands of colonic adenomas beginning in early adolescence. The average age of CRC diagnosis (if untreated) in FAP patients is 40 years; 7% develop the tumor by the age of 20 and 95% by the age of 50. Attenuated FAP is a less severe form of the disease, with an average lifetime risk of CRC of 70%. In this group, approximately 30 adenomatous polyps develop in the colon, colonic neoplasms tend to be located in the proximal colon, and cancer occurs at an older age. Gardner's syndrome and Turcot's syndrome are rare variants of FAP. In addition to polyps, Gardner's syndrome causes extra-colonic symptoms like epidermoid cysts, osteomas, dental abnormalities and/or desmoid tumors. Turcot's syndrome causes colorectal adenomatous polyps, and predisposition to developing malignant tumors of the central nervous system, such as medulloblastoma.

The genetic conditions MUTYH-associated polyposis, Peutz-Jeghers syndrome, and juvenile polyposis syndrome are other rarer syndromes that cause colon polyps, and predisposition to cancer. Patients with MUTYH-associated polyposis (MAP) develop adenomatous polyposis of the colorectum and have an 80% risk of CRC. Peutz-Jeghers and juvenile polyposis syndromes exhibit an increased risk for colorectal and other malignancies with the lifetime risk of CRC is approximately 40%.

Biliary tract cancers, also called cholangiocarcinomas, refer to those malignancies occurring in the organs of the biliary system, including pancreatic cancer, gallbladder cancer, and cancer of bile ducts. Approximately 7,500 new cases of biliary tract cancer are diagnosed each year. These cancers include about 5,000 gallbladder cancers, and between 2,000 and 3,000 bile duct cancers. The preneoplastic and neoplastic lesions of the bile duct and pancreas share analogies in terms of molecular, histological and pathophysiological features. Intraepithelial neoplasms are reported in biliary tract, as biliary intraepithelial neoplasm (BilIN), and in pancreas, as pancreatic intraepithelial neoplasm (PanIN). Both can evolve to invasive carcinomas, respectively cholangiocarcinoma (CCA) and pancreatic ductal adenocarcinoma (PDAC).

BilINs are usually encountered in the epithelium lining the extrahepatic bile ducts (EHBDs), and large intrahepatic bile ducts (IHBDs), and may also be found in the gallbladder. BilINs are microscopic lesions, with a micropapillary, pseudopapillary or flat growth pattern, involved in the process of multistep cholangiocarcinogenesis. Based on the degree of cellular and architectural atypia, BilINs have been classified into three categories: BilIN-1 (low grade dysplasia) showing the mildest changes compared to non-neoplastic epithelium of the bile ducts; BilIN-2 (intermediate grade dysplasia) with increased nuclear atypia and focal anomalies of cellular polarity as compared to BilIN-1; BilIN-3 (high grade dysplasia or carcinoma in situ), which are usually identified in proximity of cholangiocarcinoma areas.

About 30,000 new cases of pancreatic cancer are diagnosed in the United States each year. Because the early symptoms are vague, and there are no screening tests to detect it, early diagnosis is difficult. The pancreatic intraepithelial neoplasm (PanINs) is defined as a microscopic flat or micropapillary noninvasive lesions. These lesions are frequently less than 5 mm in size, and considered the most common malignant precursors of pancreatic ductal adenocarcinoma (PDAC). A lower proportion of cases of PDAC also originate from the intraductal papillary mucinous neoplasms of the pancreas (IPMNs) and mucinous cystic neoplasms (MCNs). PanINs have also been classified, according to the degree of cellular and architectural atypia, into low grade (previously classified as PanIN-1 and PanIN-2) with mild-moderate cytological atypia and basally located nuclei, and high grade (previously classified PanIN-3) with severe cytological atypia, loss of polarity and mitoses.

Inflammatory bowel disease (IBD) is a group of nonspecific chronic inflammatory diseases of the gut, which includes Crohn's disease (CD), ulcerative colitis (UC) and indeterminate colitis. The pathogenesis of IBD remains unclear, and it is characterized by long-lasting and relapsing intestinal inflammation. The incidence of UC in the United States is estimated to be between 9 and 12 per 100,000 persons with a prevalence of 205 to 240 per 100,000 persons (Tally et al., Am J Gastroenterol. 106 Suppl 1:S2-S25 (2011)). The etiology of UC is unknown. However, abnormal immune responses to contents in the gut, including intestinal microbes, are thought to drive disease in genetically predisposed individuals (Geremia et al., Autoimmun Rev. 13:3-10 (2014)). Colitis-associated colorectal cancer (CACC) is one of the most serious complications of inflammatory bowel disease (IBD), particularly in ulcerative colitis (UC); it accounts for approximately 15% of all-causes mortality among IBD patients. Because of worse prognosis and higher mortality in CACC than in sporadic CRC, early CACC detection is crucial.

Crohn's disease is marked by inflammation of the gastrointestinal (GI) tract. The inflammation can appear anywhere in the GI tract from the mouth to the anus. People with the disease often experience ups and downs in symptoms. They may even experience periods of remission. The length of diagnostic delay can represent an issue for at least a proportion of patients with Crohn's disease (CD). However, Crohn's is a progressive disease that starts with mild symptoms and gradually gets worse. Early diagnosis is important to help prevent bowel damage such as fistulae, abscesses, or strictures.

Irritable bowel syndrome (IBS) is a disorder which manifests as a set of chronic gastrointestinal (GI) symptoms and changes in bowel habits in the absence of evident structural and biochemical abnormalities. IBS has a global prevalence of 10-15% and is more frequent among individuals aged <50 years old. Lovell and Ford, Global Prevalence of and Risk Factors for Irritable Bowel Syndrome: A Meta-analysis, Clinical Gastroenterology and Hepatology 10:712-721 (2012). Altered bowel habits are the most commonly reported clinical feature, with the syndrome predominantly associated with constipation (IBS-C), diarrhoea (IBS-D) or a mixture of both conditions (IBS-M). In addition, patients with IBS often experience abdominal pain, which can be provoked by emotional stress or eating and is usually alleviated by the passing of stool. A diagnosis of IBS is confirmed according to the latest version of the Rome criteria based on the clinical experience and consensus of a committee of multinational experts.

Barrett's esophagus is a condition in which tissue that is similar to the lining of intestine replaces tissue lining esophagus. People with Barrett's esophagus may develop esophageal adenocarcinoma. The exact cause of Barrett's esophagus is unknown, but gastroesophageal reflux disease (GERD) increases the risk developing Barrett's esophagus.

Diagnosis, and specifically early diagnosis is critical for preventing mortality and morbidity in individuals suffering from precancerous lesions, GI tract cancers, ulcerative colitis, Crohn's disease, Barrett's esophagus, irritable bowel syndrome and/or irritable bowel disease.

In various aspects, the present invention provides a genetically engineered microorganism useful in the detection of the mislocalized and/or aberrantly expressed cell surface molecules in the gastrointestinal tract, and thereby diagnose, prognose, or evaluate a disease condition. The genetically engineered microorganism disclosed herein comprises a gene encoding a surface protein, wherein the surface protein specifically interacts with one or more cell membrane receptor(s), wherein the one or more cell membrane receptor(s) are not exposed to the luminal side of epithelial cells of normal gastrointestinal tissue and/or epithelial tissue lining the bile duct, pancreatic duct, or common bile duct, etc.; and wherein the one or more cell membrane receptor(s) are exposed to the luminal side of epithelial cells of diseased gastrointestinal tissue and/or epithelial tissue lining the bile duct, pancreatic duct, or common bile duct, etc. In this aspect, in some embodiments, the surface protein promotes binding and invasion of epithelial cells of diseased gastrointestinal tissue and/or epithelial tissue lining the bile duct, pancreatic duct, or common bile duct, etc. by the genetically engineered microorganism disclosed herein. The microorganism also comprises one or more gene(s) encoding the secretable biomarker, which is operably linked to a promoter. In some embodiments, the microorganism may be non-pathogenic and/or harbors at least one auxotrophic mutation. In some embodiments, the at least one auxotrophic mutation includes a deletion, inactivation, or decreased expression or activity of a gene involved in the synthesis of a metabolite (e.g., a non-genetically encoded amino acid) required for cell wall synthesis. In exemplary embodiments, the gene is required for synthesis of D-alanine or diaminopimelic acid. Such auxotrophic mutations provide a means for selection for the engineered microorganism, and also facilitate lysis of the microorganism once inside the mammalian cell.

In some embodiments, the genetically engineered microorganism of the present disclosure delivers a nucleic acid to diseased epithelial cells (target cells). The one or more gene(s) encoding the secretable biomarker may include one or more sequence element(s) operably linked to the detection marker genes that control the expression of the secretable biomarker. The sequence element may control and regulate the transcription, transcript stability, translation, protein stability, cellular localization, and/or secretion of the detection marker. In some embodiments, the sequence element may prevent expression of the detection marker by the genetically engineered microorganism. In alternative embodiments, the sequence element may allow expression (transcription and/or translation) of the detection marker by the genetically engineered microorganism.

In some embodiments, the genetically engineered microorganism of the present disclosure delivers a DNA molecule (e.g. a plasmid DNA, which is also referred to herein as a payload plasmid) to diseased epithelial cells (target cells). In some embodiments, the payload plasmid is present in multiple copies (ranging from about 1 to about 300 copies, from about 20 to about 50 copies, from about 2 to about 10 copies, or from about 5 to about 10 copies) per cell, or is a single copy plasmid. Copy number depends on the particular genetic characteristics of the plasmid. In some embodiments, the payload plasmid harbors one or more gene(s) encoding the secretable biomarker. In some embodiments, the one or more gene(s) encoding the secretable biomarker is operably linked to a mammalian promoter. In some embodiments, the one or more gene(s) encoding the secretable biomarker comprises a microbial repressor binding site(s) to inhibit bacterial transcription. In some embodiments, the one or more gene(s) encoding the secretable biomarker comprises intron(s), where removal of the introns is necessary for functional expression of the detection marker. In some embodiments, the one or more gene(s) encoding the secretable biomarker comprises microbial transcription terminator(s).

In some embodiments, the bacteria express a T7 RNA polymerase (T7RNAP) encoded by a T7RNAP gene, and harbor a gene encoding a detection marker disclosed herein under the control of a T7 promoter. In some embodiments, the T7RNAP is integrated on the bacterial chromosome. In some embodiments, the T7RNAP is present on a plasmid. In some embodiments, the T7RNAP is controlled by an inducible promoter (e.g. araBAD or lacUV5 promoters). In these embodiments, the bacteria express mRNA encoding the detection marker and/or the detection marker. In some embodiments, these bacteria deliver mRNA encoding the detection marker to diseased epithelial cells. In these embodiments, the mRNA encoding the detection marker that is delivered to diseased epithelial cells comprises an internal ribosome entry site (IRES). In some embodiments, these bacteria deliver the detection marker protein to diseased epithelial cells. In these embodiments, the detection marker that is delivered to diseased epithelial cells becomes fluorescent upon contact with a cellular metabolite.

Without being bound by theory, it is believed that the sequence element(s) that are optionally present in the gene encoding the secretable biomarker present on the payload plasmid DNA (e.g. a mammalian promoter, microbial repressor binding sites (e.g. operators), and introns) allow production of the secretable biomarker in mammalian cells, while preventing the expression of the secretable biomarker in the genetically engineered microorganism. Therefore, in some embodiments, the genetically engineered microorganism provides a true readout of the presence of diseased epithelial cells (target cells), without background expression in the genetically engineered microorganism. Accordingly, in some embodiments, the gene encoding the secretable biomarker may be operably linked to a mammalian promoter. In some embodiments, the mammalian promoter directs GI tract epithelial cell-specific expression and/or specific expression in epithelium of the ducts that are connected with gastrointestinal tissue (e.g., epithelial tissue lining the bile duct, pancreatic duct, and common bile duct). Illustrative examples of suitable mammalian promoters that direct GI tract epithelial cell-specific expression are the MUC2 gene promoter, T3b gene promoter, intestinal fatty acid binding protein gene promoter, lysozyme gene promoter and villin gene promoter. In some embodiments, the mammalian promoter directs an inducible GI tract epithelial cell-specific expression. In some embodiments, RPL38 promoter is used for specific expression in ductal epithelium of the pancreas. Illustrative example of suitable inducible mammalian promoter may be a cytochrome P450 promoter element that is transcriptionally up-regulated in response to a lipophilic xenobiotic such as β-napthoflavone. In some embodiments, the inducible mammalian promoter is regulatable by tetracycline, cumate, or an estrogen. In some embodiments, the inducible mammalian promoter may be a Tet-On or Tet-Off promoter. Accordingly, in some embodiments, the gene encoding the secretable biomarker may be inducible and/or repressible, and optionally controlled by delivering the inducer or repressor to the patient.

The microbial repressor binding sites, which are optionally present in the gene encoding the secretable biomarker repress the expression of the gene encoding the secretable biomarker in bacteria, while exerting no such repressive effect in mammalian cells. In some embodiments, the repressor sequence may be selected from one or more lac operator(s), one or more ara operator(s), one or more trp operator(s), one or more SOS operator(s), one or more integration host factor (IHF) binding sites, one or more histone-like protein HU binding sites, and a combination of two or more thereof.

The microbial transcription termination site(s) cause premature termination of the transcription of the gene encoding the secretable biomarker in the genetically engineered microorganism, without causing premature termination of the transcription of the gene encoding the secretable biomarker in mammalian cells. In some embodiments, the gene encoding the secretable biomarker comprises a rho-independent microbial transcription termination site. In some embodiments, the gene encoding the secretable biomarker comprises a 5′ untranslated region, the 5′ untranslated region comprises a rho-independent microbial transcription termination site. In some embodiments, the rho-independent microbial transcription termination site comprises a short hairpin followed by a run of 4-8 Ts (e.g. TTTTTT and TTTTT). Illustrative rho-independent microbial transcription termination sites are T7 terminator, rrnB terminator, and TO terminator.

In alternative embodiments, the genetically engineered microorganism of the present disclosure may deliver an mRNA molecule encoding secretable biomarker to the diseased epithelial cells (target cells). Accordingly, in these embodiments, the gene encoding the secretable biomarker may be operably linked to a microbial promoter. In some embodiments, the microorganism delivers an mRNA encoding the secretable biomarker to the cytoplasm of diseased epithelial cells, and which are capable of translating the mRNA to produce the encoded secretable marker. In some embodiments, the gene encoding the secretable biomarker comprises an internal ribosome entry site(s) (IRES). In these embodiments, the internal ribosome entry site promotes translation of the mRNA molecule delivered by the microorganism. In some embodiments, the mRNA sequence that is delivered comprises an element that imparts stability on the mRNA molecule. Non-limiting examples of the elements that impart stability on the mRNA molecule include 5′ hairpin structures and 3′poly A tails.

Accordingly, in these embodiments, the gene encoding the secretable biomarker may be operably linked to a microbial promoter. Illustrative examples of suitable microbial promoter include a natural promoter of any chromosomal gene, plasmid gene, or bacteriophage gene that functions in a microorganism (e.g. E. coli). In some embodiments, the microbial promoter may be a synthetic promoter derived from a promoter consensus sequence. In some embodiments, the microbial promoter may be an inducible promoter. Illustrative examples of suitable inducible microbial promoter are araBAD promoter and lac promoter. Accordingly, in some embodiments, the gene encoding the secretable biomarker may be inducible and/or repressible, and optionally controlled by delivering the inducer or repressor to the patient. In some embodiments, the promoter is a T7 or T3 promoter, and the engineered microorganism expresses a T7 or T3 RNA polymerase gene to support expression of the secretable marker.

An internal ribosome entry site (IRES) is an RNA element that allows for translation initiation in a cap-independent manner. In some embodiments, the internal ribosome entry site (IRES) may be selected from an IRES from encephalomyocarditis virus (EMCV), an IRES from hepatitis C virus (HCV), and an IRES from cricket paralysis virus (CrPV). In some embodiments, the internal ribosome entry site(s) present in the gene encoding the secretable biomarker allow for the production of the secretable biomarker in mammalian cells using an mRNA produced in the genetically engineered microorganism.

An intron(s), which is optionally present in the gene encoding the secretable biomarker prevents the functional expression of the secretable biomarker in bacteria, while allowing expression of the gene encoding the secretable biomarker in mammalian cells, irrespective of whether the mRNA encoding the secretable biomarker may be transcribed in the genetically engineered microorganism or a mammalian cell. In some embodiments, the intron may be a spliceosomal intron. In some embodiments, the intron creates a frameshift or a premature stop codon in an unspliced mRNA encoding the secretable biomarker. Therefore, in some embodiments, the genetically engineered microorganism provides a true readout of the presence of diseased epithelial cells (target cells), without background expression of the secretable biomarker protein in the genetically engineered microorganism.

In any of the embodiments disclosed herein, the gene encoding the secretable biomarker optionally further comprises a sequence element selected from Kozak sequences, 2A peptide sequences, mammalian transcription termination sequences, polyadenylation sequences (pA), leader sequences for protein secretion and a combination of any two or more thereof.

The Kozak sequence is a nucleic acid motif that functions as the protein translation initiation site in most eukaryotic mRNA transcripts. The Kozak sequence present in the gene encoding the secretable biomarker improves correct translation initiation. In some embodiments, the Kozak sequence has the following nucleotide sequence: 5′-(GCC)GCCRCCAUGG-3′.

The 2A peptides, where present, function by preventing the synthesis of a peptide bond between the glycine and proline residues found at the end of the 2A peptides, and that the 2A peptides allow production of equimolar levels of multiple proteins from the same mRNA. The 2A peptides become attached to C-terminus upstream protein, while the downstream protein starts with a proline. In some embodiments, the 2A peptide is selected from E2A ((GSG)QCTNYALLKLAGDVESNPGP), F2A ((GSG)VKQTLNFDLLKLAGDVESNP GP), P2A ((GSG)ATNFSLLKQAGDVEENPGP), and T2A ((GSG)EGRGSLLTCGDVEE NPGP). In some embodiments, the GSG sequence (which is included in the parentheses) may be optionally present.

The polyadenylation sequences (pA) cause addition of a polyA tail to mRNA, which is important for the nuclear export, translation, and stability of mRNA. The mammalian transcription termination sequences terminate transcription and promote the addition of polyA tail. In some embodiments, the gene encoding the secretable biomarker comprises a sequence element that is both a mammalian transcription termination sequence and a polyadenylation sequence. In some embodiments, the sequence element that may be both a mammalian transcription termination sequence and a polyadenylation sequence is selected from a SV40 terminator, hGH terminator, BGH terminator, and rbGlob terminator.

In some embodiments, the gene encoding the secretable biomarker comprises codon usage optimized for mammalian expression.

In some embodiments, the genetically engineered microorganism delivers a one or more nucleic acid(s) encoding secretable biomarker to the diseased epithelial cells (target cells). In these embodiments, the diseased epithelial cells (target cells) express the secretable biomarker, allowing their detection. In some embodiments, the diseased epithelial cells (target cells) secrete the secretable biomarker, rendering the secretable biomarker detectable in blood, serum, or plasma. In some embodiments, the secretable biomarker is excreted in a bodily fluid such as mucus, urine, and saliva, rendering the secretable biomarker detectable in the bodily fluid.

In some embodiments, the secretable biomarker is secretable by epithelial cells of gastrointestinal tissue. Accordingly, in these embodiments, the secretable biomarker is synthesized and sorted in the secretory pathway in mammalian cells that express the secretable biomarker. In some embodiments, the secretable biomarker comprises an ER signal sequence, optionally located at or near the N-terminus. In some embodiments, the ER signal directs the ribosomes that are synthesizing the secretable biomarker to the rough ER. In some embodiments, the secretable biomarker comprises a native signal peptide. Additionally, or alternatively, in some embodiments, the secretable biomarker comprises a heterologous signal sequence. In some embodiments, the heterologous signal sequence is selected from the signal sequence of interleukin-2, CD5, the Immunoglobulin Kappa light chain, trypsinogen, serum albumin, and prolactin. In some embodiments, the Gaussia luciferase (Glue) comprises a native signal sequence. Additionally, or alternatively, the Gaussia luciferase (Gluc) comprises a heterologous signal sequence. In some embodiments, the βhCG comprises a native signal sequence. Additionally, or alternatively, the βhCG comprises a heterologous signal sequence.

In some embodiments, the secretable biomarker is an enzyme, a peptide hormone, or a protein or peptide antigen. In some embodiments, the secretable biomarker is a secreted protein (or a subunit of peptide therefrom) selected from alkaline phosphatase, a human chorionic gonadotropin, a human carcinoembryonic antigen (CEA), colon cancer secreted protein 2 (CCSP-2), Cathepsin B, a Gaussia luciferase (Gluc), a Metridia luciferase (MLuc), a subunit thereof, a fragment thereof, and a combination of any two or more thereof. In some embodiments, the secretable biomarker is detected in a biological sample such as blood, serum, plasma, mucus, urine, and saliva, and thereby detecting the presence of the diseased epithelial cells.

In some embodiments, the secretable biomarker is measured by using an enzymatic assay (e.g., where the secretable biomarker has enzymatic activity) or an immunoassay. In these or other embodiments, the secretable biomarker is measured in the sample using an assay selected from agglutination, amperometry, atomic absorption spectrometry, atomic emission spectrometry, circular dichroism (CD), chemiluminescence, colorimetry, dipstick assay, lateral flow immunoassay, fluorimetry, electrophoretic assay, enzymatic assay, enzyme linked immunosorbant assay (ELISA), gas chromatography, gravimetry, high pressure liquid chromatography (HPLC), immunoassay, immunofluorometric enzyme assay, mass spectrometry, nuclear magnetic resonance, radioimmunoassay, spectrometry, titrimetry, western blotting, or a combination of any two or more thereof.

In some embodiments, when the secretable biomarker is detected in the biological sample, additional testing, (without limitation, e.g., colonoscopy) is indicated.

In some embodiments, the expression of the secretable biomarker may be detected in a biopsy sample using a technique selected from reverse transcription-polymerase chain reaction (RT-PCR), immunohistochemistry, fluorescent in situ hybridization (FISH, including mRNA fluorescent in situ hybridization (RNA-FISH)), and chromogenic in situ hybridization (CISH). In some embodiments, the expression of the secretable biomarker may be used for localizing diseased epithelial or gastrointestinal (GI) tissue and/or epithelium of the ducts that are connected with gastrointestinal tissue (e.g., epithelial tissue lining the bile duct, pancreatic duct, and common bile duct).

In some embodiments, the secretable biomarker is marker is a human chorionic gonadotropin (hCG). hCG is an hormone composed of two subunits: the alpha and beta subunits. In some embodiments, human chorionic gonadotropin may be an a subunit of human chorionic gonadotropin (αhCG). In some embodiments, human chorionic gonadotropin may be a p subunit of human chorionic gonadotropin (βhCG). hCG is primarily catabolized by the liver, although about 20% is excreted in the urine. The beta subunit is degraded in the kidney to make a core fragment which is measured by urine hCG tests. The pattern of excretion of hCG in urine throughout pregnancy is well established. Excretion of hCG in saliva has also been reported. Accordingly, in some embodiments, the hCG is measured using blood, serum, plasma, urine, and/or saliva.

In some embodiments, the hCG (e.g. βhCG) is optimized for distribution in body fluids (e.g. decreased or increased excretion, and increased stability). In some embodiments, the hCG is optimized for distribution in the blood. In some embodiments, the hCG is optimized for distribution in the urine. Accordingly, in some embodiments, the hCG is optimized for increased excretion (e.g., in urine or feces). In alternative embodiments, the hCG (e.g., βhCG) is optimized for decreased excretion, leading to accumulation of the hCG in the blood. In some embodiments, the hCG is optimized for increased stability (e.g., improved protein folding and/or stability against proteolysis) leading to increased accumulation. In exemplary embodiment, the hCG (e.g., βhCG) is optimized for increased stability and decreased excretion, leading to accumulation in the blood. In another exemplary embodiment, the hCG (e.g., βhCG) has increased stability and increased excretion, leading to accumulation in the urine.

In some embodiments, the hCG is measured using one anti-hCG antibody, or a fragment thereof. In some embodiments, the hCG is measured using two anti-hCG antibodies, or fragment(s) thereof. In some embodiments, the two anti-hCG antibodies, or fragment(s) thereof bind different epitopes. In some embodiments, the hCG is measured using an immunometric assay such as an enzyme-linked immunosorbent assay (ELISA). In some embodiments, the hCG is measured using an ELISA selected from direct ELISA, indirect ELISA, sandwich ELISA, and competitive ELISA. In some embodiments, any of the commercially available hCG detection kits may be used.

In an illustrative embodiment, the method for detecting diseased epthelial tissue comprises (i) administering to the target epithelial tissue (such as target tissue of the gastrointestinal tract) of a subject in need thereof, a genetically engineered microorganism of any of the embodiments disclosed herein comprising an exogenous gene encoding an hCG or a fragment thereof; (ii) obtaining a biological sample from the subject selected from blood, serum, plasma, urine and saliva; and (iii) measuring the hCG or a fragment thereof in the biological sample obtained from the subject. In any of the embodiments disclosed herein, the detection is performed using an agglutination assay, chemiluminescence, colorimetry, fluorimetry, a quantitative immunoassay, a dipstick assay, lateral flow immunoassay, enzyme linked immunosorbant assay (ELISA), or a combination of any two or more thereof.

In some embodiments, when the hCG or a fragment thereof is detected in the biological sample, additional testing, (without limitation, e.g., colonoscopy) is indicated.

In some embodiments, the expression of hCG or a fragment thereof may be detected in a biopsy sample using a technique selected from reverse transcription-polymerase chain reaction (RT-PCR), immunohistochemistry, fluorescent in situ hybridization (FISH, including mRNA fluorescent in situ hybridization (RNA-FISH)), and chromogenic in situ hybridization (CISH). In some embodiments, the expression of hCG may be used for localizing diseased epithelial or gastrointestinal (GI) tissue and/or epithelium of the ducts that are connected with gastrointestinal tissue (e.g., epithelial tissue lining the bile duct, pancreatic duct, and common bile duct).

In some embodiments, the secretable biomarker is marker is an alkaline phosphatase. In some embodiments, the alkaline phosphatase is secreted alkaline phosphatase (SEAP). Alkaline phosphatases are normally membrane-bound, thus not secreted. Without being bound by theory, introduction of a termination codon in the sequence encoding the membrane anchoring domain may yield a fully active secreted alkaline phosphatase. Lowe, “Site-specific Mutations in the COOH Terminus of Placental Alkaline Phosphatase: A Single Amino Acid Change Converts a Phosphatidylinositol-glycan-anchored Protein to a Secreted Protein” Journal of Cell Biology, 116(3): 799-807 (1992).

In some embodiments, the alkaline phosphatase (without limitation, e.g., SEAP) may be assayed from blood sample. In some embodiments, the secreted alkaline phosphatase is measured using an enzymatic assay. In some embodiments, a chromogenic, fluorogenic, luminogenic and/or substrate may be used in the assay. Illustrative chromogenic alkaline phosphatase substrates include, but are not limited to, 5-bromo-4-chloro-3-indolyl phosphate (BCIP), p-nitrophenyl phosphate (pNPP)), and 4-Chloro-2-methylbenzenediazonium/3-Hydroxy-2-naphthoic acid 2,4-dimethylanilide phosphate. Illustrative luminogenic alkaline phosphatase substrates include, but are not limited to, dioxetane phosphates (e.g., adamantyl dioxetane phenyl phosphate and chloro-5-substituted adamantyl-1,2-dioxetane phosphate (CSPD)), Illustrative fluorogenic alkaline phosphatase substrates include, but are not limited to, 4-Methylumbelliferyl phosphate disodium salt (MUP), and 6,8-difluoro-7-hydroxy-4-methylcoumarin phosphate.

In some embodiments, a substrate selected from p-nitrophenyl phosphate (pNPP), 4-Methylumbelliferyl phosphate disodium salt (MUP), and chloro-5-substituted adamantyl-1,2-dioxetane phosphate (CSPD) may be used to detect the alkaline phosphatase (without limitation, e.g., SEAP). In some embodiments, the enzymatic assay may use a colorimetric, fluorimetric and/or chemiluminiscent readout. In an illustrative embodiment, p-nitrophenyl phosphate (pNPP) may be used as the substrate and the alkaline phosphatase (without limitation, e.g., SEAP) may be assayed from a biological sample, using a colorimetric readout. In another illustrative embodiment, 4-methylumbelliferyl phosphate disodium salt (MUP) may be used as the substrate and the alkaline phosphatase (without limitation, e.g., SEAP) may be assayed from a biological sample using a fluorimetric readout. In yet another illustrative embodiment, chloro-5-substituted adamantyl-1,2-dioxetane phosphate (CSPD) may be used as the substrate and the alkaline phosphatase (without limitation, e.g., SEAP) may be assayed from a biological sample using a chemiluminiscent readout.

In an illustrative embodiment, the method for detecting diseased epithelial or gastrointestinal (GI) tissue and or diseased epithelium of the ducts that are connected with gastrointestinal tissue (e.g., epithelial tissue lining the bile duct, pancreatic duct, and common bile duct) comprises (i) administering to target epithelial cells (such as target cells of the gastrointestinal tract) of a subject in need thereof, a genetically engineered microorganism of any of the embodiments disclosed herein comprising an exogenous gene encoding an alkaline phosphatase; (ii) obtaining a biological sample from the subject selected from blood, serum, or plasma; and (iii) measuring the alkaline phosphatase in the biological sample obtained from the subject. In any of the embodiments disclosed herein, the detection is performed using chemiluminescence, colorimetry, fluorimetry, a quantitative immunoassay, lateral flow immunoassay, enzyme linked immunosorbant assay (ELISA), or a combination of any two or more thereof.

In some embodiments, when the alkaline phosphatase is detected in the biological sample, additional testing, (without limitation, e.g., colonoscopy) is indicated.

In some embodiments, the expression of the alkaline phosphatase may be detected in a biopsy sample using a technique selected from reverse transcription-polymerase chain reaction (RT-PCR), immunohistochemistry, fluorescent in situ hybridization (FISH, including mRNA fluorescent in situ hybridization (RNA-FISH)), and chromogenic in situ hybridization (CISH). In some embodiments, the expression of the alkaline phosphatase may be used for localizing diseased epithelial or gastrointestinal (GI) tissue and/or epithelium of the ducts that are connected with gastrointestinal tissue (e.g., epithelial tissue lining the bile duct, pancreatic duct, and common bile duct).

In some embodiments, the secretable biomarker is marker is a luciferase. In some embodiments, the luciferase is Gaussia luciferase (Gluc), a Metridia luciferase (MLuc), a subunit thereof, a fragment thereof. Gluc is naturally secreted from mammalian cells in an active form. Moreover, Gluc is excreted at least in urine. Accordingly, in some embodiments, the Gluc may be measured using blood, serum, plasma, urine, and/or saliva.

In some embodiments, the luciferase is Gaussia luciferase (Gluc) or a derivative thereof. In some embodiments, the luciferase is measured using an enzymatic assay. In some embodiments, the luciferase is measured using a luciferin as a substrate. The luciferin, without limitation, may be firefly luciferin, Latia luciferin, bacterial luciferin, dinoflagellate luciferin, vargulin, or foxfire. In some embodiments, ATP may be a cofactor. In some embodiments, the luciferin is coelenterazine. In some embodiments, the luciferase is measured using a luminescence readout. In some embodiments, the biological sample is selected from blood, urine, and saliva.

Gaussia luciferase (Gluc) is a small luciferase with numerous disulfide bonds. Gluc is stable in serum, however, it not active in E. coli cytoplasm because the bacterial environment does not allow the disulfide bonds to form. Accordingly, in some embodiments, expression of Glue is indicative of production and secretion of Gluc by diseased human cells. In some embodiments, the gene encoding Gluc comprises an intron to ensure production and secretion of Gluc by diseased human cells.

Gluc does not require any cofactors for activity (e.g., ATP) and catalyzes the oxidation of the substrate coelenterazine in a reaction that leads to emission of blue light (480 nm). Accordingly, in some embodiments, the Gluc may be measured using luminescence. In other embodiments, the Gluc may be measured using an immunoassay such as a quantitative immunoassay, a lateral flow immunoassay, an enzyme-linked immunosorbent assay (ELISA).

In some embodiments, the Gluc is optimized for distribution in body fluids (e.g., decreased or increased excretion, and increased stability). In some embodiments, the Gluc is optimized for distribution in the blood. In some embodiments, the Gluc is optimized for distribution in the urine. Accordingly, in some embodiments, the Gluc is optimized for increased excretion (e.g., in urine or feces). In alternative embodiments, the Gluc (e.g., βGluc) is optimized for decreased excretion, leading to accumulation of the Gluc in the blood. In some embodiments, the Gluc is optimized for increased stability (e.g., improved protein folding and/or stability against proteolysis) leading to increased accumulation. In exemplary embodiment, the Gluc is optimized for increased stability and decreased excretion, leading to accumulation in the blood. In another exemplary embodiment, the Gluc has increased stability and increased excretion, leading to accumulation in the urine.

In an illustrative embodiment, the method for detecting diseased epithelial or gastrointestinal (GI) tissue or diseased epithelium of the ducts that are connected with gastrointestinal tissue (e.g., epithelial tissue lining the bile duct, pancreatic duct, and common bile duct) comprises (i) administering to target epithelial cells (such as target cells of the gastrointestinal tract) of a subject in need thereof, a genetically engineered microorganism of any of the embodiments disclosed herein comprising an exogenous gene encoding an Gluc or a fragment thereof; (ii) obtaining a biological sample from the subject selected from blood, serum, plasma, urine and saliva; and (iii) measuring the Gluc or a fragment thereof in the biological sample obtained from the subject. In any of the embodiments disclosed herein, the detection is performed using a luminescence assay.

In some embodiments, when the Glue or a fragment thereof is detected in the biological sample, additional testing, (without limitation, e.g., colonoscopy) is indicated. In some embodiments, the expression of Gluc or a fragment thereof may be detected based on luminescence, during colonoscopy. In these embodiments, coelenterazine may be administered to the subject before or during colonoscopy.

In some embodiments, the expression of Gluc or a fragment thereof may be detected in a biopsy sample using a technique selected from reverse transcription-polymerase chain reaction (RT-PCR), immunohistochemistry, fluorescent in situ hybridization (FISH, including mRNA fluorescent in situ hybridization (RNA-FISH)), and chromogenic in situ hybridization (CISH). In some embodiments, the expression of Gluc may be used for localizing diseased gastrointestinal (GI) tissue and/or diseased epithelium of the ducts that are connected with gastrointestinal tissue (e.g., epithelial tissue lining the bile duct, pancreatic duct, and common bile duct).

In some embodiments, the secretable biomarker is marker is human carcinoembryonic antigen (CEA). CEA is naturally expressed in some normal adult tissues, and tumor expressing the same self-antigen. In some embodiments, the secretable biomarker is marker is colon cancer secreted protein 2 (CCSP2). CCSP2 expression is generally absent in normal body tissues, but is induced in colon adenomas and colon cancers. In some embodiments, the secretable biomarker is cathepsin B. Cathepsin B belongs to a family of lysosomal cysteine proteases and plays an important role in intracellular proteolysis. It is found both in serum and urine.

The genetically engineered microorganism disclosed herein comprises one or more gene(s) encoding a surface protein, wherein the surface protein specifically interacts with one or more cell membrane receptor(s) that are specifically exposed on the luminal side of epithelial cells of diseased gastrointestinal tissue. In this aspect, in some embodiments, the surface protein promotes the binding and invasion specifically of epithelial cells of diseased tissue by the genetically engineered microorganism.

In some embodiments, the surface protein is an invasin, or a fragment thereof. In some embodiments, the surface protein is the invasin is selected from Yersinia enterocolitica invasin, Yersinia pseudotuberculosis invasin (SEQ ID NO: 1), Salmonella enterica PagN, Candida albicans Als3 and E. coli intimin.

In some embodiments, the surface protein is invasin (SEQ ID NO: 1) or YadA (Yersinia enterocolitica plasmid adhesion factor). Alternative surface proteins include Rickettsia invasion factor RickA (actin polymerization protein), Legionella RaIF (guanine exchange factor), one or more Neisseria invasion factors (e.g. NadA (Neisseria adhesion/invasion factor), OpA and OpC (opacity-associated adhesions)), Listeria InlA and/or InlB, one or more of Shigella invasion plasmid antigens (e.g., IpaA, IpaB, IpaC, IpgD, IpaB-IpaC complex, VirA, and IcsA), one or more of Salmonella invasion factor (e.g., SipA, SipC, SpiC, SigD, SopB, SopE, SopE2, and SptP), Staphylococcus FnBPA and/or FnBPB, one or more Streptococcus invasion factor (ACP, Fba, F2, Sfb1, Sfb2, SOF, and PFBP), and/or Porphyromonas gingivalis FimB (integrin binding protein fibriae). In some embodiments, the surface protein comprises an active fragment of one or more of invasin, YadA, RickA, RaIF, NadA, OpA, OpC, InlA, InlB, IpaA, IpaB, IpaC, IpgD, IpaB-IpaC, VirA, IcsA, SipA, SipC, SpiC, SigD, SopB, SopE, SopE2, SptP, FnBPA, FnBPB, ACP, Fba, F2, Sfb1, Sfb2, SOF, PFBP, and FimB. In some embodiments, the fragment is expressed on the surface of the engineered microorganism disclosed herein, e.g., on an adhesion scaffold. In some embodiments, the surface protein is a fusion protein of invasin and intimin. In some embodiments, intimin-invasin was made by replacing the three C-terminal one or more domains of intimin (e.g., D1, D2 and D3) is fused with the C-terminal domain of invasin.

In some embodiments, the surface protein is a type III secretion system or a component thereof. In some embodiments, the surface protein comprises a peptide or protein that specifically binds to the surface of cancerous and pre-cancerous cells, optionally wherein the protein is selected from a peptide, a leptin, an antibody, or a fragment thereof (e.g., sdAb, also known as Nanobody® and an scFv fragment). In some embodiments, the surface protein comprises one or more of leptins, antibodies, or fragments thereof. Illustrative examples of fragments of antibodies are single-domain antibody (sdAb, also known as Nanobody®) or scFv fragments, without limitation, including a camelid nanobody. In some embodiments, the leptin, the antibody, or the fragment thereof are displayed on as a fusion protein with a microbial surface protein. In some embodiments, the microbial surface protein is selected from invasin, intimin and adhesin. In some embodiments, the peptide or camelid nanobody is selected for its ability to bind to cancer cell surface receptors. In some embodiments, the surface protein comprises a peptide or protein that specifically binds to mislocalized proteins in cancerous tissues or precancerous lesions (polyps or adenomas), tears and erosions (Barett's Esophagus), or inflammatory diseases.

By virtue of the identity of the surface protein, the genetically engineered microorganism disclosed herein may mimic the affinity of the native surface protein. In some embodiments, the genetically engineered microorganism disclosed herein may specifically bind to one or more of oral epithelial cells, buccal epithelial cells of the tongue, pharyngeal epithelial cells, mucosal epithelial cells, endothelial cells of the stomach, intestinal epithelial cells, colon epithelial etc.

In some embodiments, the genetically engineered microorganism disclosed herein comprises a second exogenous gene encoding a lysin that lyses the endocytotic vacuole, and thereby contributes to pore-formation, breakage or degradation of the phagosome. In some embodiments, the lysin is a cholesterol-dependent cytolysin. In some embodiments, the lysin is selected from the group consisting of listeriolysin O, ivanolysin O, streptolysin, perfringolysin, botulinolysin, leukocidin and a mutant derivative thereof. In some embodiments, the lysin is listeriolysin O, or a mutant derivative thereof. In some embodiments, the mutant derivative of listeriolysin O is a derivative that is not secreted, and remains in the cytoplasm (e.g., SEQ ID NO: 8). In some embodiments, the mutant derivative of listeriolysin O is a derivative that is secreted in the periplasm (e.g., SEQ ID NO: 2). In some embodiments, the mutant derivative of listeriolysin O is a derivative that is secreted outside the outer membrane.

The genetically engineered microorganism of the present technology may be derived from any non-pathogenic microorganism, such as the non-pathogenic microorganisms that are normal flora of human GI tract or the microorganisms that are generally recognized as safe for human consumption via foods like yogurts, cheeses, breads and the like. In some embodiments, the genetically engineered microorganism of any one of the embodiments disclosed herein may be derived from a microorganism selected from Lactobacillus, Bifidobacterium, Saccharomyces, Enterococcus, Streptococcus, Lactococcus, Pediococcus, Leuconostoc, Bacillus, and Escherichia coli. Illustrative species that are suitable for genetically engineering microorganism of any one of the embodiments disclosed herein include Bacillus coagulans, Bifidobacterium adolescentis, Bifidobacterium animalis, Bifidobacterium bifidum, Bifidobacterium breve, Bifidobacterium essencis, Bifidobacterium faecium, Bifidobacterium infantis, Bifidobacterium lactis, Bifidobacterium longum, Bifidobacterium longum subsp. infantis, Bifidobacterium pseudolungum, Lactobacillus acidophilus, Lactobacillus boulardii, Lactobacillus breve, Lactobacillus brevis, Lactobacillus bulgaricus, Lactobacillus casei, Lactobacillus delbrueckii ssp. Bulgaricus, Lactobacillus fermentum, Lactobacillus gasseri, Lactobacillus helveticus, Lactobacillus paracasei, Lactobacillus plantarum, Lactobacillus reuteri, Lactobacillus rhamnosus, Lactobacillus rhamnosus GG, Lactobacillus salivarius, Lactococcus lactis, Streptococcus thermophilus, Pediococcus acidilactici, Enterococcus faecium, Leuconostoc, Carnobacterium, Proprionibacterium, Saccharomyces boulardii, and Escherichia coli.

In some embodiments, the genetically engineered microorganism of any one of the embodiments disclosed herein may be derived from a probiotic Escherichia coli strain such as Escherichia coli Nissle 1917, Escherichia coli Symbioflor2 (DSM 17252), Escherichia coli strain AO 34/86, Escherichia coli 083 (Colinfant). In some embodiments, the genetically engineered microorganism of any one of the embodiments disclosed herein is derived from Escherichia coli Nissle 1917. The complete genome sequence of Escherichia coli Nissle 1917 is known. Reister et al., J Biotechnol. 187:106-7 (2014).

In some embodiments, the genetically engineered microorganism of any one of the embodiments disclosed herein is an Escherichia coli Nissle 1917 or a derivative thereof. Escherichia coli Nissle 1917 contains two naturally occurring, stable, cryptic plasmids pMUT1 (GenBank Accession No. MW240712) and the plasmid pMUT2 (GenBank Accession No. CP023342). In some embodiments, the Escherichia coli Nissle 1917 or the derivative thereof harbors a plasmid pMUT1 and/or a plasmid pMUT2, and/or one or more derivative thereof. In some embodiments, the Escherichia coli Nissle 1917 or the derivative thereof is cured of the plasmid pMUT1 and/or the plasmid pMUT2.

In some embodiments, the gene encoding the surface protein is integrated in the genome of the microorganism. Additionally, or alternatively, the gene encoding the lysin is integrated in the genome of the microorganism. In some embodiments, the gene encoding the surface protein, and the gene encoding the lysin are integrated at a single genomic site. In some embodiments, the single genomic site is an integration site of a bacteriophage, and/or an integration site of a plasmid. Thus, in some embodiments, the gene encoding the secretable biomarker may be inserted at the bacteriophage lambda integration site attB.

In some embodiments, the gene encoding the surface protein is integrated on a plasmid of the microorganism. Additionally, or alternatively, the gene encoding the lysin is integrated on a plasmid. In some embodiments, the plasmid is a naturally occurring plasmid, e.g., present in the microorganism such as Escherichia coli Nissle 1917, or is an engineered plasmid. In some embodiments, the gene encoding the surface protein, and the gene encoding the lysin are integrated on a single plasmid.

In some embodiments, the gene encoding the secretable biomarker may be inserted on a natural endogenous plasmid from the microorganism (e.g., Escherichia coli Nissle 1917, such as pMUT1, pMUT2, and/or a derivative thereof). In some embodiments, the plasmid comprises a selection mechanism, such as the complementation of an auxotrophic mutation as described herein. In some embodiments, the plasmid is a multi-copy plasmid (e.g., a high copy number plasmid), thereby providing a high nucleic acid payload upon cell invasion. In alternative embodiments, the gene encoding the surface protein is inserted on the same or different plasmid. In some embodiments, the gene encoding the lysin is inserted on the same or different plasmid.

Additionally, or alternatively, the gene encoding the secretable biomarker is integrated in the genome of the microorganism. In some embodiments, the gene encoding the surface protein, the second gene encoding the lysin and the gene encoding the secretable biomarker are integrated at a single genomic site. In some embodiments, the single genomic site is an integration site of a bacteriophage, and/or a integration site of a plasmid.

In some embodiments, the plasmid and/or the second plasmid comprises a selection mechanism. In some embodiments, the selection mechanism may not require an antibiotic for plasmid maintenance. Accordingly, in some embodiments, the selection mechanism is selected from an antibiotic resistance marker, a toxin-antitoxin system, a marker causing complementation of a mutation in an essential gene, a cis acting genetic element and a combination of any two or more thereof.

In some embodiments, the selection mechanism is a resistance marker to an antibiotic that is not used or is rarely in human or animals for therapy. In some embodiments, the selection mechanism used for selection of the plasmid and/or the second plasmid is an antibiotic resistance marker selected from kanamycin resistance gene, tetracycline resistance gene and a combination thereof. Additionally, or alternatively, in some embodiments, the selection mechanism used for selection of the plasmid and/or the second plasmid is a toxin-antitoxin system selected from a hok/sok system of plasmid R1, parDE system of plasmid RK2, ccdAB of F plasmid, flmAB of F plasmid, kis/kid system of plasmid R1, XCV2162-ptaRNA1 of Xanthomonas campestris, ataT-ataR of enterohemorragic E. coli or Klebsiella, toxIN system of Erwinia carotovora, parE-parD system of Caulobacter crescentus, fst-RNAII from Enterococcus faecalis plasmid AD1, ε-ζ system of Bacillus subtilis plasmid pSM19035 and a combination of any two or more thereof. Additionally, or alternatively, in some embodiments, the selection mechanism used for selection of the plasmid and/or the second plasmid is an essential gene encoding an enzyme involved in biosynthesis of an essential nutrient or a substrate (e.g., an amino acid) required for cell wall synthesis; and/or a house-keeping function. Exemplary amino acids required for cell wall synthesis include D-alanine and diaminopimelic acid. In some embodiments, the essential gene is selected from dapA, dapD, murA, alr, dadX, murI, dapE, thyA and a combination of any two or more thereof. In some embodiments, the essential genes are a combination of alr and dadX (both of which encode for alanine racemases). In some embodiments, the essential genes are a combination of alr and dadX, and the plasmid is selected using a functional alr gene (alr*, e.g., a wild type alr gene) as a selection marker. In some embodiments, the plasmid and/or the second plasmid is selected by complementation of the alr and dadX mutations by a functional alr gene present on the plasmid and/or the second plasmid. In some embodiments, the house-keeping function is selected from infA, a gene encoding a subunit of an RNA polymerase, a DNA polymerase, an rRNA, a tRNA, a cell division protein, a chaperon protein, and a combination of any two or more thereof. Additionally, or alternatively, in some embodiments, the selection mechanism used for selection of the plasmid and/or the second plasmid is a cis acting genetic element such as ColE1 cer locus or par from pSC101.

In some embodiments, when the genetically engineered microorganism delivers an mRNA molecule the gene encoding the secretable biomarker to the diseased epithelial cells (target cells), the gene encoding the secretable biomarker is integrated in the genome of the genetically engineered microorganism. In some embodiments, when the genetically engineered microorganism delivers an mRNA molecule the gene encoding the secretable biomarker to the diseased epithelial cells (target cells), the gene encoding the secretable biomarker is present on a plasmid.

In alternative embodiments, when the genetically engineered microorganism delivers a DNA molecule (e.g., a plasmid) comprising the gene encoding the secretable biomarker to the diseased epithelial cells (target cells), the gene encoding the secretable biomarker is present on a plasmid. In some embodiments, the plasmid comprising the gene encoding the secretable biomarker further comprises at least one binding site for a DNA binding protein. In some embodiments, a binding site for a DNA binding protein forms an array of multiple adjacent binding sites for a DNA binding protein. In some embodiments, the DNA binding protein comprises one or more nuclear localization signal(s) (NLS). In these embodiments, the DNA binding protein binds the DNA molecule (e.g., a plasmid) and promotes the nuclear translocation of the DNA molecule (e.g., a plasmid) via the one or more nuclear localization signal(s) (NLS). In some embodiments, the NLS is SV40 T antigen NLS sequence (KKKRKV). In some embodiments, the DNA binding protein is NFκB. In some embodiments, the microorganism comprises a gene encoding the DNA binding protein comprises one or more nuclear localization signal(s) (NLS). Without being bound by theory, it is believed that the DNA binding protein comprising one or more nuclear localization signal(s) binds the at least one binding site for the DNA binding protein on the plasmid comprising the one or more gene(s) encoding at least one detection marker and promotes nuclear translocation of the plasmid via the action of one or more nuclear localization signal(s). Thus, in these embodiments, the diseased epithelial cells express the at least one detection marker from the DNA molecule (e.g., a plasmid) delivered by the microorganism, thereby allowing their detection.

In some embodiments, the DNA binding protein that binds to the at least one binding site is expressed by the microorganism. In an illustrative embodiment, a plasmid comprising the gene encoding the secretable biomarker further comprises an array of lac operators or tet operators, and the microorganism overexpresses lac repressor or tet repressor comprising a nuclear localization signal. In other embodiments, the DNA binding protein that binds to the at least one binding site is expressed by the diseased epithelial cells. In an illustrative embodiment, a plasmid comprising the gene encoding the secretable biomarker further comprises an array of NFκB binding sites, and the diseased epithelial cells express NFκB.

In some embodiments, the gene encoding the DNA binding protein is genomically integrated, or present on a plasmid.

In some embodiments, the microorganism harbors at least one nutritional auxotrophic mutation that facilitates lysis of the microorganism inside the mammalian cell upon invasion. Such mutations include deletions, inactivations, or reduced expression or activity of genes involved in cell wall synthesis or metabolites required for cell wall synthesis, as well as alterations of other proteins such as porins. In some embodiments, the microorganism harbors a deletion or mutation in a gene selected from dapA, dapD, dapE, murA, alr, dadX, murI, thyA, aroC, ompC, and ompF. In some embodiments, the microorganism harbors a combination of dapA, alr and dadX auxotrophic mutations. In some embodiments, a plasmid is selected by complementation of the alr and dadX mutations by a functional alr gene present on the plasmid. In some embodiments, the at least one nutritional auxotrophic mutation facilitates lysis of the microorganism inside the diseased mammalian cell upon invasion. In some embodiments, the dapA auxotrophic mutation facilitates lysis of the microorganism inside the diseased mammalian cell upon invasion.

In some embodiments, about 10³ to about 10¹¹ viable genetically engineered microorganisms are administered to a subject, depending on the species of the subject, as well as the disease or condition that is being diagnosed or treated. In some embodiments, about 10⁵ to about 10⁹ viable genetically engineered microorganisms of the present disclosure are administered to a subject.

The genetically engineered microorganisms of the present disclosure may be administered between 1 and about 50 times prior to detection of the expressed marker. The genetically engineered microorganisms may be administered from about 1 to about 21, or from 1 to about 14, or from about 1 to about 7 times prior to the marker detection. The genetically engineered microorganisms may be administered starting between about 1 hour to about 2 months prior to marker detection. The administration of the genetically engineered microorganisms may be started prior to marker detection by at least about 1 hour, at least about 6 hours, at least about 12 hours, at least about 24 hours, at least about 2 days, at least about 3 days, at least about 5 days, at least about 7 days, at least about 10 days, at least about 15 days, at least about 20 days, at least about 30 days, at least about 40 days, at least about 50 days, or at least about 60 days.

The genetically engineered microorganisms of the present disclosure may be administered by any route as long as they are capable of invading their target cells upon administration and capable of delivery of their payload. The payload that the genetically engineered microorganisms of the present disclosure deliver are generally a nucleic acid molecule encoding a secretable biomarker. In some embodiments, the genetically engineered microorganism of the present technology is administered by oral and/or rectal route.

The genetically engineered microorganisms of the present disclosure are generally administered along with a pharmaceutically acceptable carrier and/or diluent. The particular pharmaceutically acceptable carrier and/or diluent employed is not critical to the present invention. Examples of diluents include a phosphate buffered saline, buffer for buffering against gastric acid in the stomach, such as citrate buffer (pH 7.0) containing sucrose, bicarbonate buffer (pH 7.0) alone (Levine et al., J. Clin. Invest. 79:888-902 (1987); and Black et al., J. Infect. Dis. 155:1260-1265 (1987)), or bicarbonate buffer (pH 7.0) containing ascorbic acid, lactose, and optionally aspartame (Levine et al., Lancet 2(8609):467-70 (1988)). Examples of carriers include proteins, e.g., as found in skim milk, sugars, e.g., sucrose, or polyvinylpyrrolidone. Typically these carriers would be used at a concentration of about 0.1-30% (w/v) but preferably at a range of 1-10% (w/v).

The pharmaceutically acceptable carriers or diluents which may be used for delivery may depend on specific routes of administration. Any such carrier or diluent can be used for administration of the genetically engineered microorganisms of the invention, so long as the genetically engineered microorganisms of the present disclosure are still capable of invading a target cell and delivering the payload that they carry to the target cells. In vitro or in vivo tests for invasiveness can be performed to determine appropriate diluents and carriers. The compositions of the invention can be formulated for oral and/or rectal administration. Lyophilized forms are also included, so long as the genetically engineered microorganisms are invasive and capable of delivering their payload upon contact with a target cell or upon administration to the subject. Techniques and formulations generally may be found in Remington's Pharmaceutical Sciences, Meade Publishing Co., Easton, Pa.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The pharmaceutical compositions provided herein may be administered rectally in the forms of suppositories, pessaries, pastes, powders, creams, ointments, solutions, emulsions, suspensions, gels, foams, sprays, or enemas. These dosage forms can be manufactured using conventional processes as described in Remington: The Science and Practice of Pharmacy, supra.

Rectal suppositories are solid bodies for insertion into rectum, which are solid at ordinary temperatures but melt or soften at body temperature to release the genetically engineered microorganisms of the present disclosure inside the rectum. Pharmaceutically acceptable carriers utilized in rectal suppositories include bases or vehicles, such as stiffening agents, which produce a melting point in the proximity of body temperature, when formulated with the pharmaceutical compositions provided herein; and antioxidants, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal suppositories may be prepared by the compressed method or molding. The typical weight of a rectal suppository is about 2 to about 3 g.

In some embodiments, the genetically engineered microorganisms of the present disclosure are administered as a single composition, or they are administered individually at the same or different times and via the same or different route (e.g., oral and rectal) of administration. In some embodiments, the genetically engineered microorganisms of the present disclosure is provided in a mixture or solution suitable for rectal instillation and comprises sodium thiosulfate, bismuth subgallate, vitamin E, and sodium cromolyn. In some embodiments, a therapeutic composition of the invention comprises, in a suppository form, butyrate, and glutathione monoester, glutathione diethylester or other glutathione ester derivatives. The suppository can optionally include sodium thiosulfate and/or vitamin E. The pharmaceutical compositions may also be formulated in rectal compositions such as suppositories or retention enemas, e.g., containing conventional suppository bases such as cocoa butter or other glycerides.

In some embodiments, the genetically engineered microorganisms of the present disclosure are formulated as an enema formulation. The enema formulation comprises a reducing agent (or any other agent having a similar mode of action). In some embodiments, an enema formulation of the invention comprises the genetically engineered microorganisms. The enema formulation can optionally comprise polysorbate-80 (or any other suitable emulsifying agent), and/or any short chain fatty acid (e.g., a five, four, three, or two carbon fatty acid) as a colonic epithelial energy source, such as sodium butyrate (4 carbons), proprionate (3 carbons), acetate (2 carbons), etc., and/or any mast cell stabilizer, such as cromolyn sodium (GASTROCROM) or Nedocromil sodium (ALOCRIL).

In some embodiments, the composition comprises from about 105 to about 109 viable genetically engineered microorganisms of the present disclosure. If the composition comprises cromolyn sodium it can be present in an amount from about 10 mg to about 200 mg, or from about 20 mg to about 100 mg, or from about 30 mg to about 70 mg. If the composition comprises polysorbate-80, it can be provided at a concentration from about 1% (v/v) to about 10% (v/v). If the composition comprises sodium butyrate it can be present in an amount of about 500 to about 1500 mg. In some embodiments, the composition suitable for administration as an enema is formulated to include genetically engineered microorganisms of the present disclosure, cromolyn sodium, and polysorbate-80. In some embodiments, the composition further comprises alpha-lipoic acid and/or L-glutamine and/or N-acetyl cysteine and/or sodium butyrate (1.1 gm).

The compositions may, if desired, be presented in a pack or dispenser device and/or a kit that may contain one or more unit dosage forms containing the active ingredient. The pack may for example comprise metal or plastic foil, such as a blister pack. The pack or dispenser device may be accompanied by instructions for administration.

In various aspects, the present invention provides a method for detecting diseased epithelial tissue, such as epithelial tissue of the gastrointestinal (GI) tissue and/or diseased epithelium of the ducts that are connected with gastrointestinal tissue (e.g., epithelial tissue lining the bile duct, pancreatic duct, and common bile duct), the method comprising (i) administering to the epithelial tissue of a subject in need thereof, a genetically engineered microorganism disclosed herein; and (ii) detecting the expression of the secretable biomarker to thereby detect the presence of diseased epithelial cells. As discussed above, the microorganism comprises an exogenous gene encoding a surface protein, wherein the surface protein specifically interacts with one or more cell membrane receptor(s), which are not exposed to the luminal side of epithelial cells of normal gastrointestinal tissue but is exposed to the luminal side of diseased epithelial cells of gastrointestinal tissue in the subject suffering from a disease. In some embodiments, the surface protein promotes binding and invasion of the microorganism in the diseased epithelial cells. The microorganism also comprises a gene encoding the secretable biomarker operably linked to a promoter.

In some embodiments, the secretable biomarker is expressed from a mammalian promoter. In some embodiments, the mammalian promoter that is active or specific for epithelial expression or GI tract epithelial cell-specific expression. In some embodiments, the mammalian promoter directs GI tract epithelial cell-specific expression and/or expression specific to epithelium of the ducts that are connected with gastrointestinal tissue (e.g., epithelial tissue lining the bile duct, pancreatic duct, and common bile duct). In these embodiments, the microorganism delivers a DNA molecule (e.g., a plasmid) to diseased epithelial cells. In some embodiments, the genetically engineered microorganism is administered via oral or rectal route. In some embodiments, the method further comprises administration of a colon cleansing agent comprising a laxative. In some embodiments, the colon cleansing agent comprising the laxative is administered prior to the administration of the microorganism.

The diseased gastrointestinal (GI) tissue may be precancerous lesion(s), a GI tract cancer, ulcerative colitis, Crohn's disease, Barrett's esophagus, irritable bowel syndrome and irritable bowel disease. Illustrative precancerous lesion(s) and GI tract cancers include squamous cell carcinoma of the anus, low-grade squamous intraepithelial lesions (LSIL) of the anus, high-grade squamous intraepithelial lesions (HSIL) of the anus, colorectal cancer, colorectal adenocarcinoma, familial adenomatous polyposis, hereditary nonpolyposis colorectal cancer, colorectal polyposis (e.g. Peutz-Jeghers syndrome, juvenile polyposis syndrome, MUTYH-associated polyposis, familial adenomatous polyposis/Gardner's syndrome, and Cronkhite-Canada syndrome), carcinoid, pseudomyxoma peritonei, duodenal adenocarcinoma, premalignant adenoma of small bowel, distal bile duct carcinomas, biliary intraepithelial neoplasm (BilIN), BilIN-1, BilIN-2, BilIN-3 or cholangiocarcinoma, pancreatic ductal adenocarcinoma (PDAC), pancreatic intraepithelial neoplasm (PanIN), PanIN-1, PanIN-2, PanIN-3, gastric carcinoma, signet ring cell carcinoma (SRCC), gastric lymphoma (MALT lymphoma), linitis plastic (Brinton's disease), and squamous cell carcinoma of esophagus and adenocarcinoma.

In some embodiments, the gastrointestinal (GI) tissue and/or epithelium of the ducts that are connected with gastrointestinal tissue (e.g., epithelial tissue lining the bile duct, pancreatic duct, and common bile duct) may be potentially diseased because the subject suffers from a precancerous lesion, cancer, ulcerative colitis, Crohn's disease, Barrett's esophagus, irritable bowel syndrome or irritable bowel disease. In some embodiments, the precancerous lesion comprises a polyp selected from sessile polyp, serrated polyp (e.g., hyperplastic polyps, sessile serrated adenomas/polyps, and traditional serrated adenoma), sessile serrated polyp, flat polyp, sub-pedunculated polyp, pedunculated polyp, and a combination thereof. In some embodiments, the polyp is a diminutive polyp. In some embodiments, the precancerous lesion comprises a biliary intraepithelial neoplasm (BilIN) selected from BilIN-1, BilIN-2, BilIN-3, and cholangiocarcinoma. In some embodiments, the precancerous lesion comprises a pancreatic intraepithelial neoplasm (PanIN) selected from PanIN-1, PanIN-2, PanIN-3 and pancreatic ductal adenocarcinoma (PDAC). In some embodiments, the precancerous lesion has a size of from about 0.05 mm to about 30 mm. In some embodiments, the precancerous lesion has a size of from less than about 0.1 mm, less than about 0.25 mm, less than about 0.5 mm, less than about 1 mm, less than about 2 mm, less than about 5 mm, less than about 8 mm, less than about 10 mm, less than about 15 mm, less than about 20 mm, less than about 25 mm, less than about 30 mm.

In some embodiments, the cancer comprises a polyp, an adenoma, or a frank cancer. In some embodiments, the cancer comprises Lynch syndrome, familial adenomatous polyposis, hereditary non-polyposis colon cancer (HNPCC), or a sporadic cancer. In some embodiments, the cancer comprises a biliary intraepithelial neoplasm (BilIN), BilIN-1, BilIN-2, BilIN-3 or cholangiocarcinoma), pancreatic intraepithelial neoplasm (PanIN), PanIN-1, PanIN-2, PanIN-3 or pancreatic ductal adenocarcinoma (PDAC).

In some embodiments, the secretable biomarker is selected from a fluorescent protein, a bioluminescent protein, a contrast agent for magnetic resonance imaging (MRI), a Positron Emission Tomography (PET) reporter, an enzyme reporter, a contrast agent for use in computerized tomography (CT), a Single Photon Emission Computed Tomography (SPECT) reporter, a photoacoustic reporter, an X-ray reporter, an ultrasound reporter, and ion channel reporters (e.g., cAMP activated cation channel), and a combination of any two or more thereof. In some embodiments, the fluorescent protein, the bioluminescent protein, the contrast agent for magnetic resonance imaging (MRI), the Positron Emission Tomography (PET) reporter, the enzyme reporter, the contrast agent for use in computerized tomography (CT), the Single Photon Emission Computed Tomography (SPECT) reporter, the photoacoustic reporter, the X-ray reporter, the ultrasound reporter, and the ion channel reporters (e.g., cAMP activated cation channel) of any of the embodiments disclosed herein may be used.

In some embodiments, the method further comprises administration of one or more substrate(s) of the at least one bioluminescent protein, one or more substrate(s) of the at least one contrast agent for use in magnetic resonance imaging, one or more PET probe(s), one or more substrate of the enzyme reporter, one or more SPECT probe(s) or a combination of any two or more thereof. In some embodiments, the administration of one or more substrate(s) of the at least one bioluminescent protein, one or more substrate(s) of the at least one contrast agent for use in magnetic resonance imaging, one or more PET probe(s), one or more substrate of the enzyme reporter, one or more SPECT probe(s) or a combination of any two or more thereof may be started prior to marker detection by at least about 1 hour, at least about 6 hours, at least about 12 hours, at least about 24 hours, at least about 2 days, at least about 3 days prior to marker detection. In some embodiments, the one or more substrate(s) of the at least one bioluminescent protein, one or more substrate(s) of the at least one contrast agent for use in magnetic resonance imaging, one or more PET probe(s), one or more substrate of the enzyme reporter, one or more SPECT probe(s) or a combination of any two or more thereof may be administered after the administration of the microorganism.

In various aspects, the present invention provides a method for evaluation, monitoring, diagnosis, and/or prognosis of a disease or disorder in a subject, the method comprising (i) administering to target epithelial tissue (e.g., of the gastrointestinal tract) of a subject in need thereof, a genetically engineered microorganism disclosed herein; and (ii) obtaining a biological sample from the subject; and (iii) measuring the secretable biomarker in the biological sample to thereby detecting the presence or absence diseased epithelial cells. As discussed above, the microorganism comprises an exogenous gene encoding a surface protein, wherein the surface protein specifically interacts with one or more cell membrane receptor(s), which are not exposed to the luminal side of epithelial cells of normal gastrointestinal tissue but is exposed to the luminal side of diseased epithelial cells of gastrointestinal tissue in the subject suffering from a disease. In some embodiments, the surface protein promotes binding and invasion of the microorganism in the diseased epithelial cells. The microorganism also comprises a gene encoding the secretable biomarker operably linked to a promoter. In some embodiments, the secretable biomarker is expressed from a mammalian promoter. In some embodiments, the mammalian promoter that is active or specific for epithelial expression (e.g., epithelium of the ducts that are connected with gastrointestinal tissue (e.g., epithelial tissue lining the bile duct, pancreatic duct, and common bile duct)) or GI tract epithelial cell-specific expression. In some embodiments, the mammalian promoter directs GI tract epithelial cell-specific expression. In these embodiments, the microorganism delivers a DNA molecule (e.g., a plasmid) to diseased epithelial cells. In some embodiments, the genetically engineered microorganism is administered via oral or rectal route. In some embodiments, the method further comprises administration of a colon cleansing agent comprising a laxative. In some embodiments, the colon cleansing agent comprising the laxative is administered prior to the administration of the microorganism. In some embodiments, the genetically engineered microorganism is non-pathogenic. In some embodiments, the genetically engineered microorganism is auxotrophic. In some embodiments, the genetically engineered microorganism is non-pathogenic and auxotrophic.

In various aspects, the present invention provides a genetically engineered microorganism for use in a method of diagnosis and/or prognosis of a disease or disorder in a subject, the method comprising (i) administering to the gastrointestinal tract of a subject in need thereof, disclosed herein; and (ii) detecting the expression of the detection marker to thereby detecting the diseased epithelial cells. As discussed above, the microorganism comprises an exogenous gene encoding a surface protein, wherein the surface protein specifically interacts with one or more cell membrane receptor(s), which are not exposed to the luminal side of epithelial cells of normal gastrointestinal tissue and/or epithelial tissue lining the bile duct, pancreatic duct, or common bile duct, etc. but is exposed to the luminal side of diseased epithelial cells of gastrointestinal tissue and/or epithelial tissue lining the bile duct, pancreatic duct, or common bile duct, etc. in the subject suffering from a disease. In some embodiments, the surface protein promotes binding and invasion of the microorganism in the diseased epithelial cells. The microorganism also comprises one or more gene(s) encoding at least one detection marker operably linked to a promoter. In some embodiments, the promoter is a mammalian promoter. In some embodiments, the mammalian promoter directs GI tract epithelial cell-specific expression. In some embodiments, the genetically engineered microorganism is administered via oral or rectal route. In some embodiments, the method further comprises administration of a colon cleansing agent comprising a laxative. In some embodiments, the colon cleansing agent comprising the laxative is administered prior to the administration of the microorganism. In some embodiments, the genetically engineered microorganism is non-pathogenic. In some embodiments, the genetically engineered microorganism is auxotrophic. In some embodiments, the genetically engineered microorganism is non-pathogenic and auxotrophic.

Disclosed herein, in various aspects, are methods of selecting a subject suffering from or suspected to be suffering from a disease for a treatment, the method comprising: (i) administering to the gastrointestinal tract of the subject a genetically engineered microorganism of any one of embodiments disclosed herein; (ii) obtaining a biological sample from the subject; and (iii) measuring the secretable biomarker in the biological sample to thereby detecting the diseased epithelial cells; and (iv) selecting the subject for treatment if expression of the secretable biomarker is observed. In some embodiments, treatable diseases include precancerous lesions, cancer, ulcerative colitis, Crohn's disease, Barrett's esophagus, irritable bowel syndrome and irritable bowel disease. In some embodiments, the treatment is surgery or administration of a therapeutic agent. In some embodiments, the surgery removes diseased tissue. In some embodiments, the therapeutic agent is selected from a chemotherapeutic agent, a cytotoxic agent, an immune checkpoint inhibitor, an immunosuppressive agent, a sulfa drug, a corticosteroid, an antibiotic and a combination of any two or more thereof.

In some embodiments, the precancerous lesion comprises a polyp selected from sessile polyp, serrated polyp (e.g., hyperplastic polyps, sessile serrated adenomas/polyps, and traditional serrated adenoma), sessile serrated polyp, flat polyp, sub-pedunculated polyp, pedunculated polyp, and a combination thereof. In some embodiments, the polyp is a diminutive polyp. In some embodiments, the polyp is a diminutive polyp. In some embodiments, the precancerous lesion comprises a biliary intraepithelial neoplasm (BilIN) selected from BilIN-1, BilIN-2, BilIN-3, and cholangiocarcinoma. In some embodiments, the precancerous lesion comprises a pancreatic intraepithelial neoplasm (PanIN) selected from PanIN-1, PanIN-2, PanIN-3 and pancreatic ductal adenocarcinoma (PDAC). In some embodiments, the precancerous lesion has a size of from about 0.05 mm to about 30 mm. In some embodiments, the precancerous lesion has a size of less than about 0.1 mm, less than about 0.25 mm, less than about 0.5 mm, less than about 1 mm, less than about 2 mm, less than about 5 mm, less than about 8 mm, less than about 10 mm, less than about 15 mm, less than about 20 mm, less than about 25 mm, or less than about 30 mm.

Additionally, or alternatively, in some embodiments, the cancer comprises a polyp, an adenoma, or a frank cancer. In some embodiments, the cancer comprises Lynch syndrome, familial adenomatous polyposis, hereditary non-polyposis colon cancer (HNPCC), or a sporadic cancer.

Also disclosed herein, in various aspects, are methods of treating a cancer in a patient. These methods comprise: (i) administering to the gastrointestinal tract of the subject a genetically engineered microorganism of any one of claims 57 to 100; (ii) detecting the expression of the secretable biomarker to thereby detecting the diseased epithelial cells; and (iii) administering a treatment if the expression of the secretable biomarker is observed. In some embodiments, the treatment is surgery or administration of a therapeutic agent. In some embodiments, the therapeutic agent is selected from the group consisting of a chemotherapeutic agent, a cytotoxic agent, an immune checkpoint inhibitor, an immunosuppressive agent, a sulfa drug, a corticosteroid, an antibiotic and a combination of any two or more thereof.

### EXAMPLES

### Example 1. Genetically Engineered Bacterial Strains and the Setup

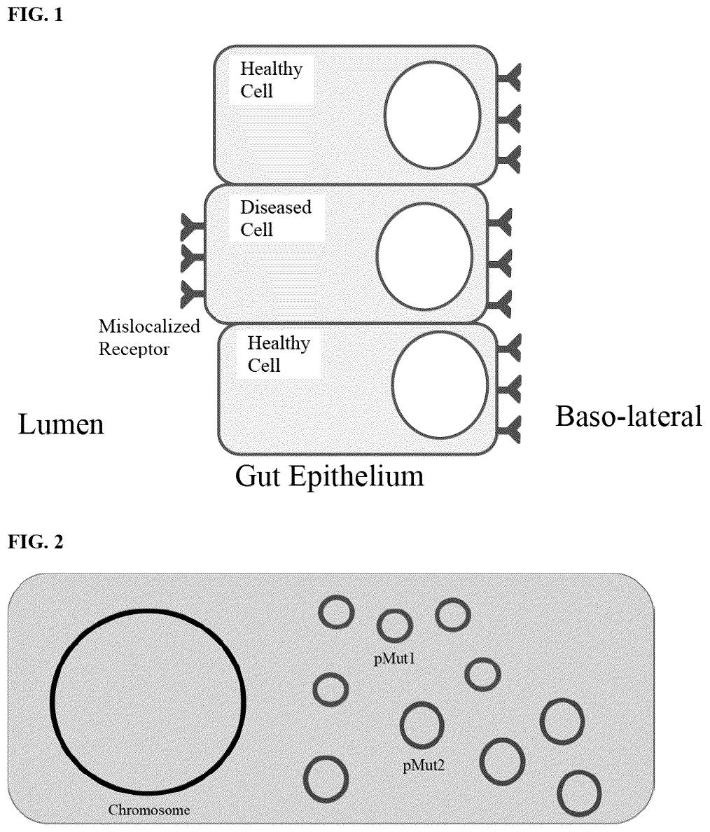

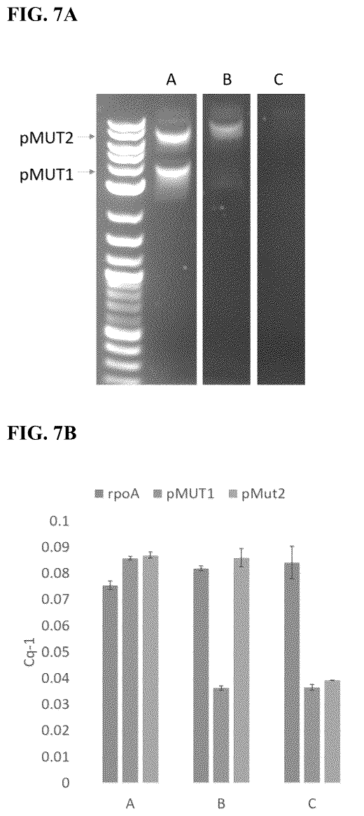

One of the objectives of this study was to construct bacterial strains that can detect diseased cells in gastrointestinal tract epithelium. As shown in FIG. 1, diseased epithelial cells exhibits mislocalized and/or expression of novel mammalian membrane receptors. The novel receptors may be the receptors that are normally not found in the cells or the receptors formed by translocations and other genomic rearrangement. Disclosed herein are bacterial strains derived from Escherichia coli Nissle 1917. As shown in FIG. 2, the strain contains a single bacterial chromosome and two extra chromosomal plasmids (pMUT1 and pMUT2). See lane A of FIG. 7.

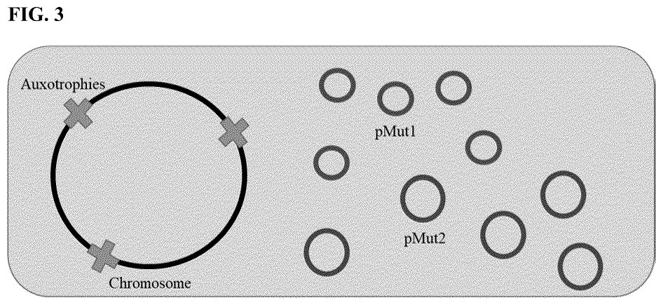

Nutritional auxotrophies were introduced (See FIG. 3) to allow containment of the bacterial strains. The nutritionally auxotrophic strains cannot reproduce in the body or environment. Moreover, the nutritional auxotrophies allow for the antibiotic free selection of the plasmids.

For bacterial containment, dapA gene, which is essential to produce diaminopimelic acid, an essential component of the bacterial cell wall, was knocked out. ΔdapA strains require diaminopimelic acid in the media for growth. For plasmid selection, alr and dadX genes were knocked out. alr and dadX are redundant alanine racemases and render the bacterial strain dependent on being supplied with the amino acid D-Alanine, which is also component of the bacterial cell wall, for growth.

All auxotrophies were generated with the well-established lambda red recombination system and done in such a way as to eliminate the antibiotic marker. Datsenko and Wanner, Proc Nat Acad Sci USA. 97(12):6640-5 (2000). As a result, the final strain is sensitive to all antibiotics that the E. coli Nissle 1917 strain is sensitive to and is expected to require the addition of diaminopimelic acid and D-alanine for growth.

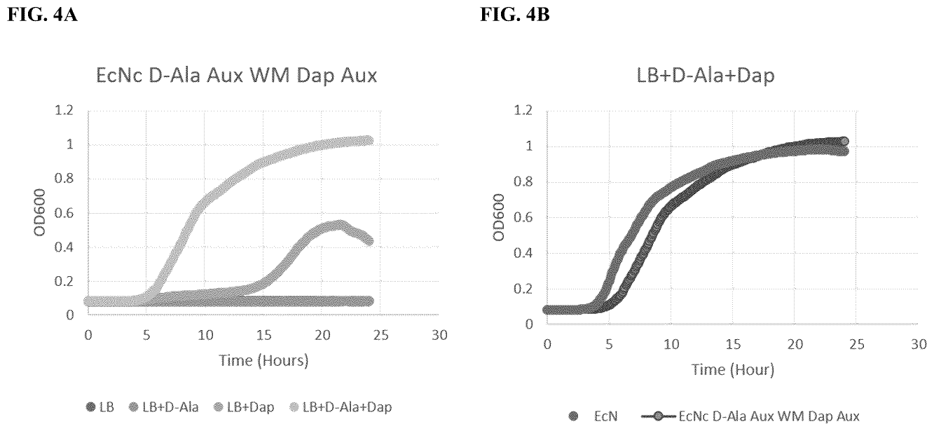

The resultant strain (E. coli Nissle 1917 ΔdapA Δalr ΔdadX) was grown in LB media supplemented with D-alanine and diaminopimelic acid. The cultures were diluted in (1) LB, (2) LB supplemented with D-alanine only, (3) LB supplemented diaminopimelic acid only, and (4) LB supplemented with D-alanine and diaminopimelic acid, incubated at 37° C., and growth was monitored. As shown in FIG. 4A, the strain only grew only when both D-alanine and diaminopimelic acid were added to the media. Further, as shown in FIG. 4B, when D-alanine and diaminopimelic acid were added to the media, the strain exhibited growth properties that were similar to that of the wild type strain.

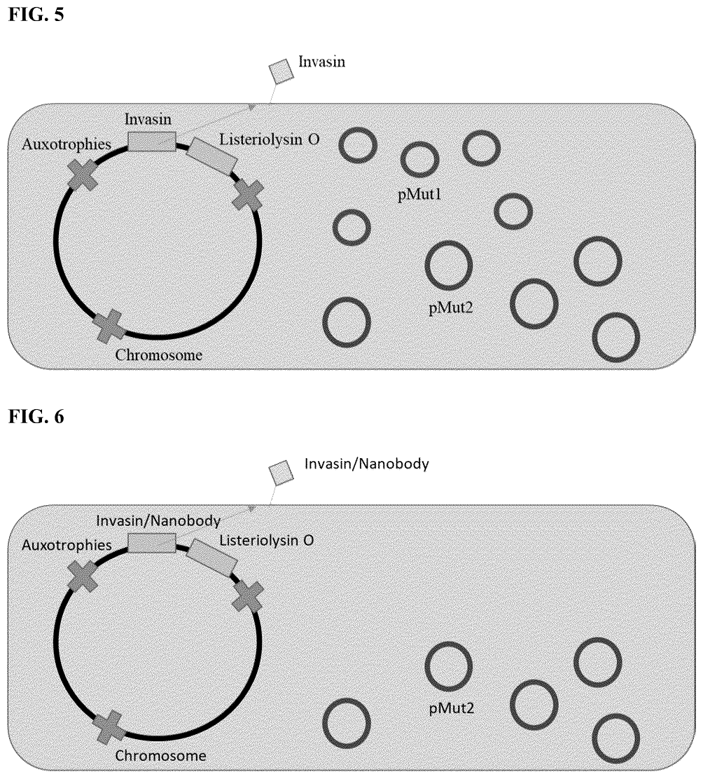

A chassis containing invasin (SEQ ID NO: 1) and listeriolysin O (SEQ ID NO: 2) was created. This invasin and listeriolysin O genes were maintained on a plasmid. A bacterial strain harboring stably integrated invasin and listeriolysin O will be constructed (FIG. 5).

Next, the plasmids pMUT1 and pMUT2 were cured using standard procedures (FIG. 6). FIG. 7A shows an agarose gel showing results of an experiment conducted to cure plasmids pMUT1 and pMUT2 from an E. coli Nissle 1917 (EcN) derivative. Wild type E. coli Nissle 1917 (EcN) was transformed with a curing plasmid and passaged in the presence of 5 mg/ml ampicillin. Plasmid preparations from wild type E. coli Nissle 1917 (EcN) (lane A), E. coli Nissle 1917 (EcN) cured of pMUT1 (lane B), and E. coli Nissle 1917 (EcN) cured of pMUT1 and pMUT2 (lane C). Expected locations of plasmids pMUT1 and pMUT2 are shown. FIG. 7B shows the results of a quantitative PCR experiment to confirm that the plasmids have been cured. Data labels are the same as in FIG. 7A.

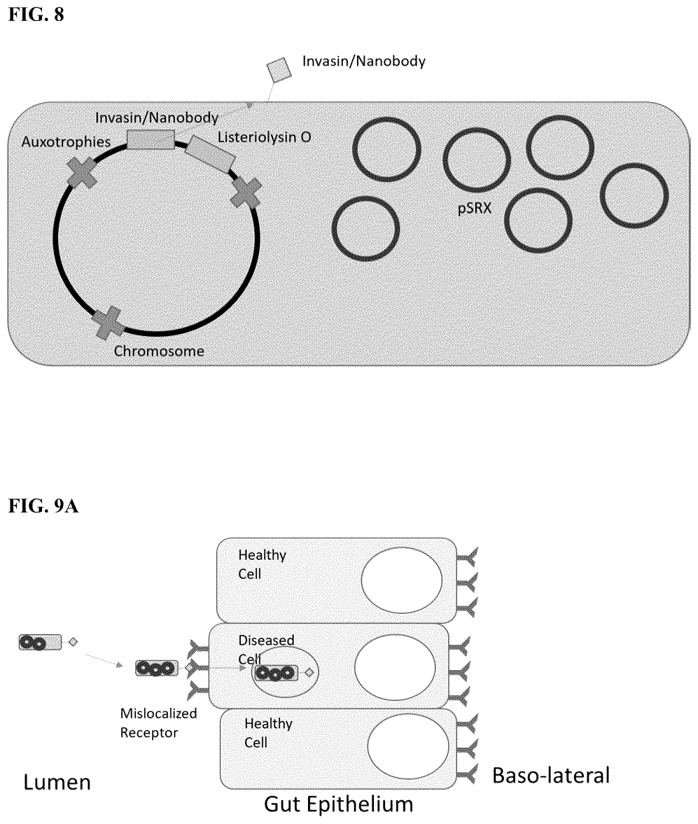

A pMUT1-based plasmid vector having a non-antibiotic selection was constructed. Summarily, E. coli alr gene (SEQ ID NO: 5) was used as selection in dapA, alr, dadX triple deletant derivative of E. coli Nissle 1917. GFP gene was cloned into the resulting plasmid selected using alr. This plasmid was named pSRX. FIG. 8 shows a schematic representation of an embodiment of the genetically engineered bacterium of present disclosure. This strain is an E. coli Nissle 1917 (EcN) derivative harboring one or more auxotrophic mutation(s) (shown by X), further having genes encoding surface protein and listeriolysin O integrated in the genome. This strain does not contain the plasmid pMUT1, but contains the plasmid pSRX, a pMUT1-based derivative, which is selected using complementation of an auxotrophic mutation as the selection mechanism. Plasmid pSRX also carries a detection marker, which is exemplified herein by GFP.

### Example 2. In Vitro Detection of Colorectal Carcinoma Cells

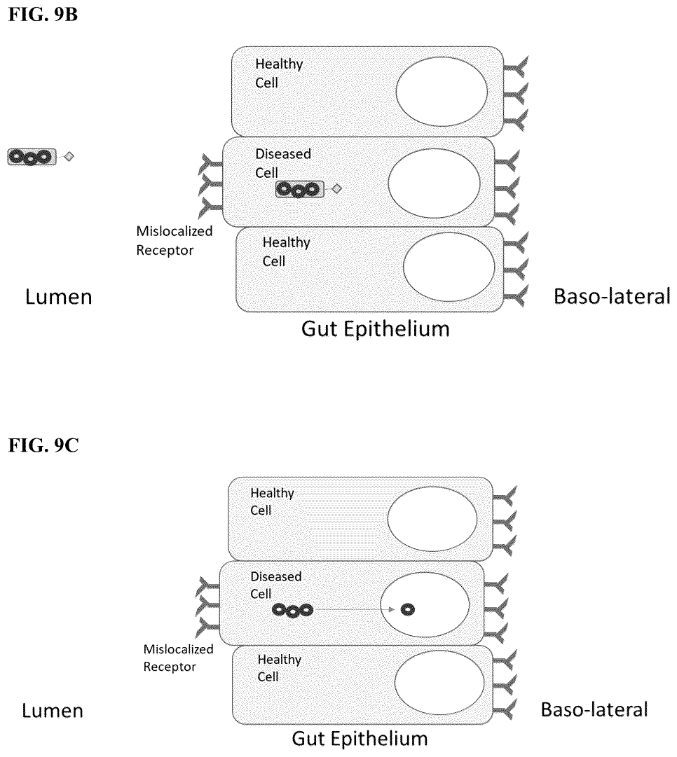

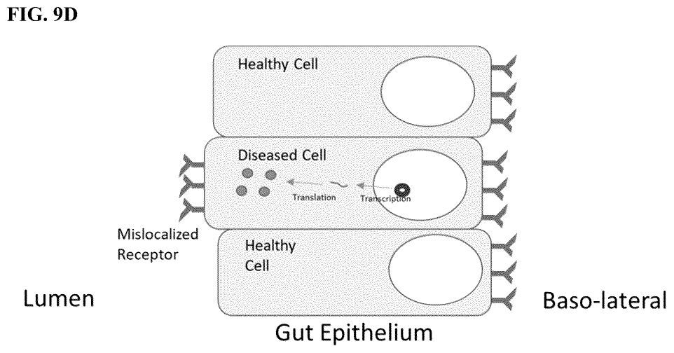

Bacteria of the current disclosure can specifically detect diseased cells. Without being bound by theory, it is hypothesized that detection of diseased cells proceeds through four distinct steps. As shown in FIG. 9A, the genetically engineered microorganisms of the current disclosure bind to diseased epithelial cells through mislocalized receptors, and undergo internalization. Upon internalization, as shown in FIG. 9B, bacteria undergo lysis due to the dapA attenuation mutation, which causes a defect in cell wall synthesis. Listeriolysin O (LLO, or Hly; SEQ ID NO: 2) is then released and lyses phagosome or is naturally exported from the E. coli strain. As shown in FIG. 9C, the plasmid carrying secretable biomarker undergoes nuclear localization, which can occur naturally or be guided by binding of a protein that includes a nuclear localization signal. As shown in FIG. 9D, the plasmid carrying secretable biomarker drives the expression of the secretable biomarker in the diseased epithelial cells of GI tract and/or epithelium of the ducts that are connected with gastrointestinal tissue (e.g., epithelial tissue lining the bile duct, pancreatic duct, and common bile duct).

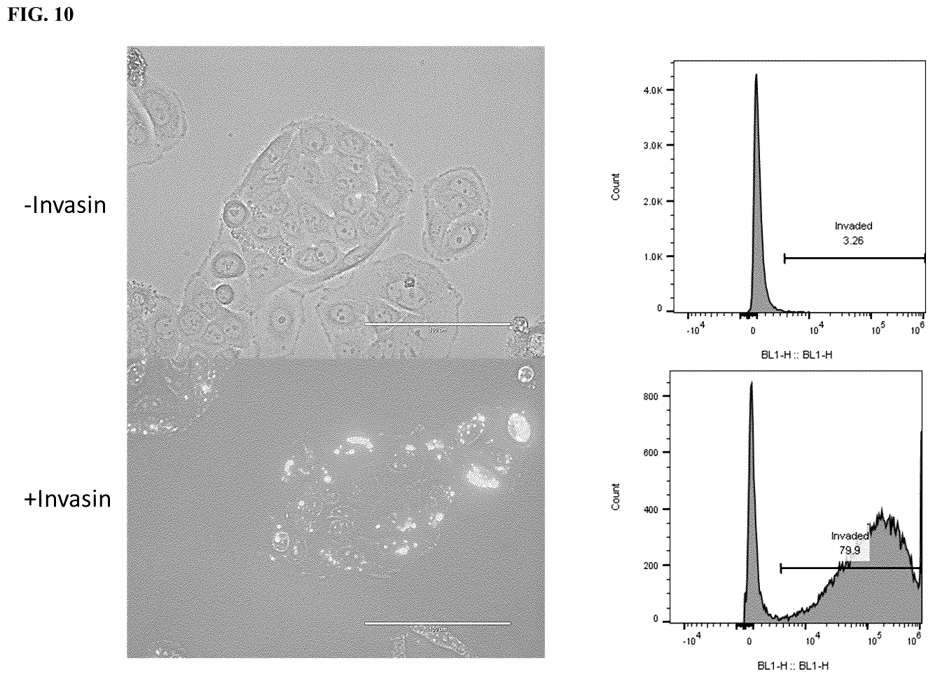

To test this scheme, a strain containing invasion machinery was constructed. The invasion machinery consists of a bacterial surface protein that binds to a protein on the mammalian cell surface and facilitating endocytosis of the bacterium. The initial bacterial surface protein tested was the inv gene from Yersinia pseudotuberculosis coding for the protein invasin (SEQ ID NO: 1). Invasin binds to integrins on the surface of mammalian cells and facilitates endocytotsis. The strain is E. coli Nissle 1917 harboring a pMUT1 derived plasmid that expresses inv under control of the ProD constitutive promoter. The plasmid also included a GFP gene under control of the same ProD promoter to make the bacteria easily visible and distinguishable from the mammalian cells. The bacteria from this strain were coincubated with SW480 (a colorectal cancer derived cell line) for one hour followed by washing away of extracellular bacteria. SW480 cells were visualized by fluorescence microscopy, removed from the plate, and then analyzed by flow cytometry to identify the portion of the SW480 cells that were successfully invaded by the bacterial strain. As shown in FIG. 10, by SW480 colorectal cancer cells were only invaded by bacterial cells expressing the invasion gene. These results demonstrate that the genetically engineered microorganisms of the current technology can invade cancer cells in vitro.

The invasion machinery comprising genes encoding invasin (inv) and listeriolysin O (hly) which allows the bacteria to escape the endocytotic vacuole will be integrated onto the bacterial chromosome at the lambda phage integration site. The integration will occur in such a way as to allow elimination of the antibiotic selection after integration. FIG. 5 shows a schematic representation this embodiment of the genetically engineered bacterium E. coli Nissle 1917 (EcN) strain. This strain optionally harbors one or more auxotrophic mutation(s) such as dapAΔ, alrΔ, and dadXΔ (shown by X).

### Example 3. In Vitro Detection of Cancer Cells Using Gaussia Luciferase (Gluc)

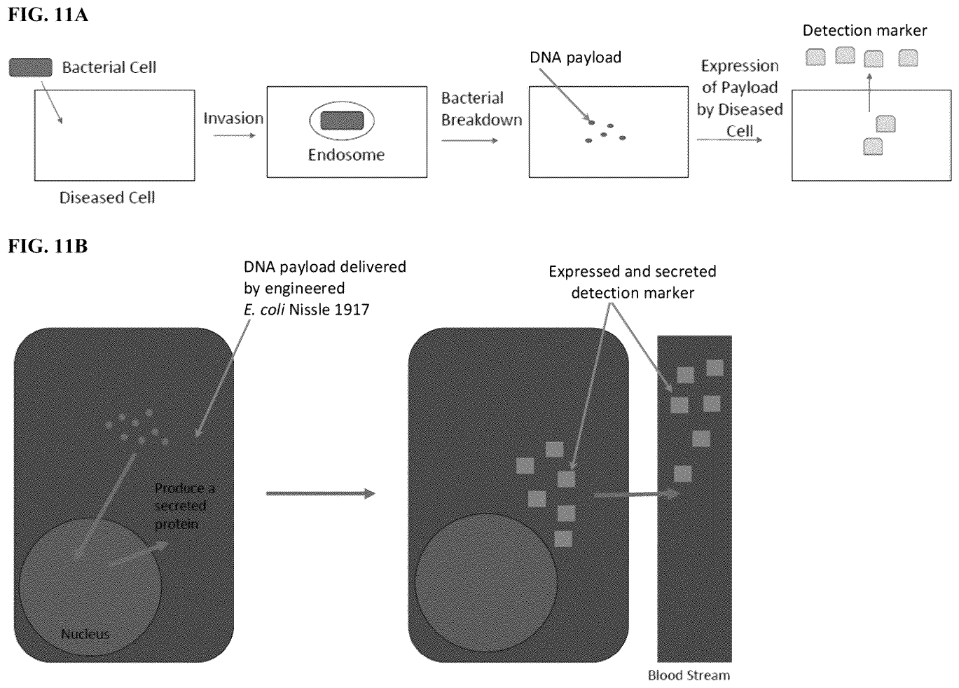

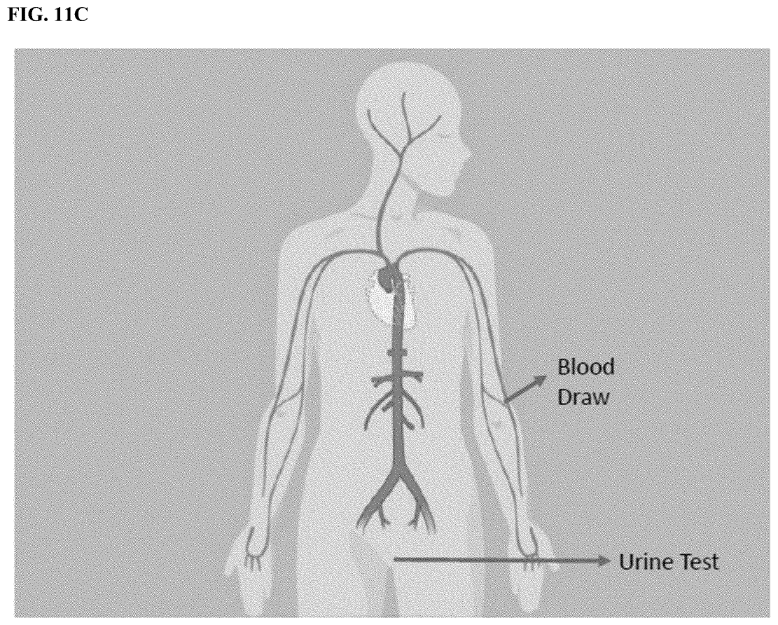

The engineered bacteria disclosed herein bind diseased cells via the surface proteins they express. For example, the engineered bacteria bind to p 1-integrin via invasin expressed on the surface. This interaction leads to bacterial invasion (FIG. 11A). Without being bound by theory, the bacteria undergo lysis. Listeriolysin O (LLO) mediates lysis of the phagosome and DNA payload is delivered (FIG. 11A). The cancer cells then express and secrete the detection marker (FIG. 11A). The detection marker may become bloodborne (FIG. 11B), making the detection of diseased cells amenable via a simple blood or urine test (FIG. 11C).

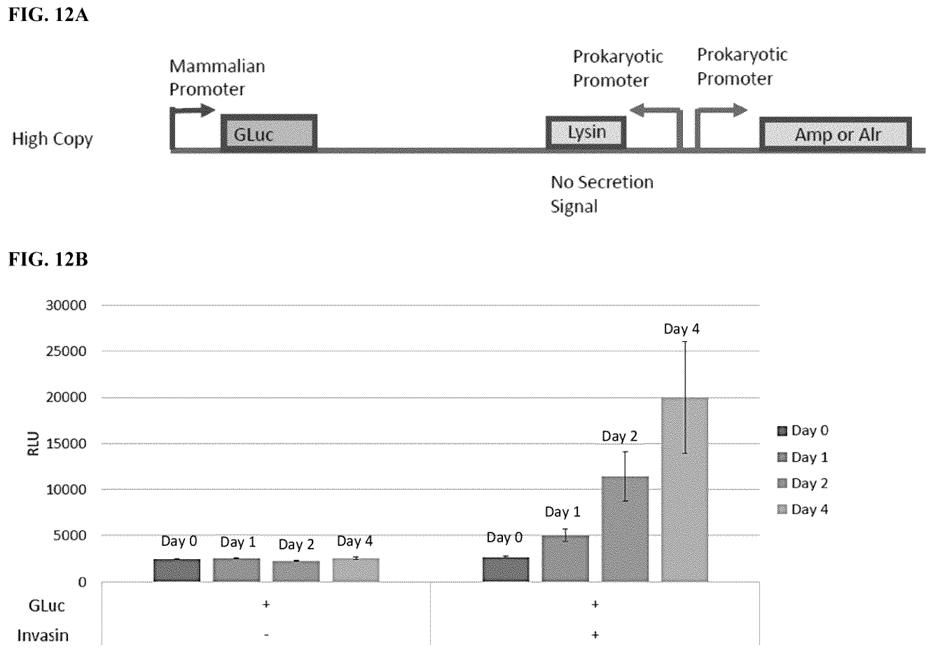

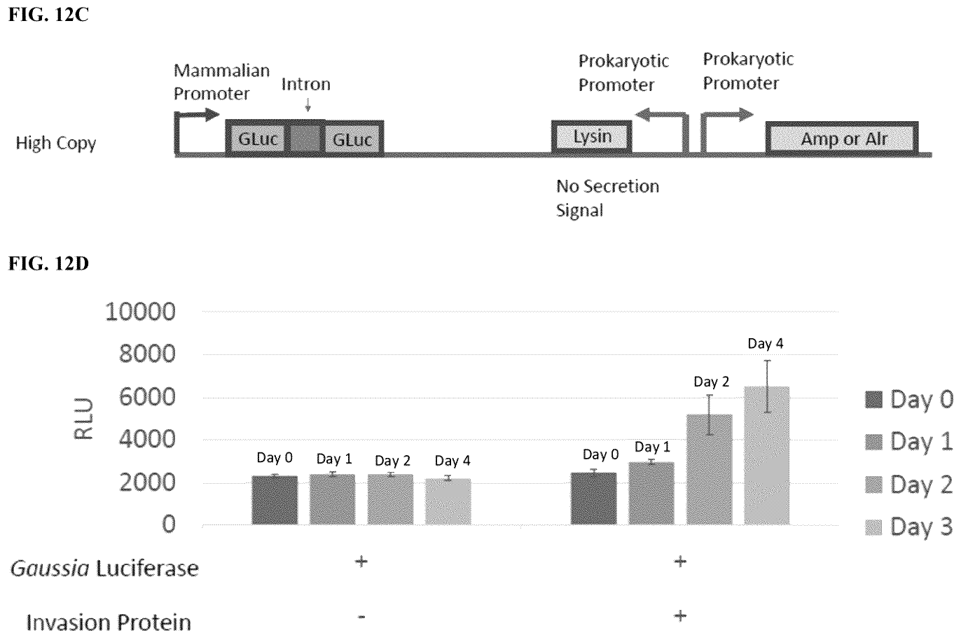

An invasin⁺, listeriolysin⁺, Gluc⁺E. coli Nissle 1917 dapAΔ experimental strain having the invasin gene integrated in the chromosome (SEQ ID NO: 7) and a high copy plasmid carrying harboring a Gaussia luciferase a (Gluc) gene under control of a mammalian promoter (SEQ ID NO: 3), and listeriolysin O (Lysin; SEQ ID NO: 2) under control of a bacterial promoter and a selection marker was constructed (FIG. 12A). An invasin⁻, listeriolysin⁺, Gluc⁺E. coli Nissle 1917 dapAΔ control strain (lacking invasin gene but carrying the plasmid having the Glue and lysin genes) was also constructed. To detect the cancer cells, the invasin⁺, listeriolysin⁺, Gluc⁺ experimental bacteria and the invasin⁻, listeriolysin⁺, Gluc⁺ control bacteria were seeded onto SW480 cells at identical multiplicities of infection. A sample containing both bacteria and media was removed and Gluc levels were measured as background level of Gluc (day 0). After 1 hour incubation, cells were washed with PBS and antibiotics were added to kill any extracellular bacteria. Cells were incubated for additional 1, 2 or 4 days and samples of media were removed for the measurement of Gluc. As shown in FIG. 12B, the invasin⁺, listeriolysin⁺, Gluc⁺ experimental bacteria produced a luminescent signal that increased from day 0 to day 1, to day 2 and to day 4. In contrast, the invasin⁻, listeriolysin⁺ Gluc⁺ control bacteria did not produce an increase in luciferase signal.

To confirm that the Glue was made by the cancer cells, an invasin⁺, listeriolysin⁺, Gluc-intron⁺E. coli Nissle 1917 dapAΔ strain having invasin gene on a low copy plasmid derived from pMUT1 and a high copy plasmid carrying harboring a Gaussia luciferase a (Gluc) gene having an intron and under control of a mammalian promoter (SEQ ID NO: 4), and listeriolysin O (lysin; SEQ ID NO: 2) under control of a bacterial promoter and a selection marker was constructed (FIG. 12C). A similar invasin⁻, listeriolysin⁺, Gluc-intron⁺E. coli Nissle 1917 dapAΔ control strain was also constructed. The invasin⁺, listeriolysin⁺, Gluc-intron⁺ bacteria and the invasin⁻, listeriolysin⁺, Gluc-intron⁺ control bacteria were seeded onto SW480 cells at identical multiplicities of infection. A sample containing both bacteria and media was removed and Gluc levels were measured as background level of Gluc (day 0). After 1 hour incubation, cells were washed with PBS and antibiotics were added to kill any extracellular bacteria. Cells were incubated for additional 1, 2 or 4 days and samples of media were removed for the measurement of Gluc. As shown in FIG. 12D, the invasin⁺, listeriolysin⁺, Gluc-intron⁺ experimental bacteria produced a luminescent signal that increased from day 0 to day 1, to day 2 and to day 4. In contrast, the invasin-, listeriolysin⁺ Gluc-intron⁺ control bacteria did not produce an increase in luciferase signal.

These results demonstrate that the cancer cells produced and secreted Gluc upon delivery of the DNA payload by the engineered bacteria disclosed herein.

Collectively, these results demonstrate that the engineered bacteria disclosed herein delivered a DNA payload to cancer cells, which resulted in production and secretion of Gluc by the cancer cells and thereby facilitated the detection of the cancer cells.

### Example 4. In Vitro Detection of Colorectal Carcinoma Cells Using Human β-Chorionic Gonadotropin (hCG)

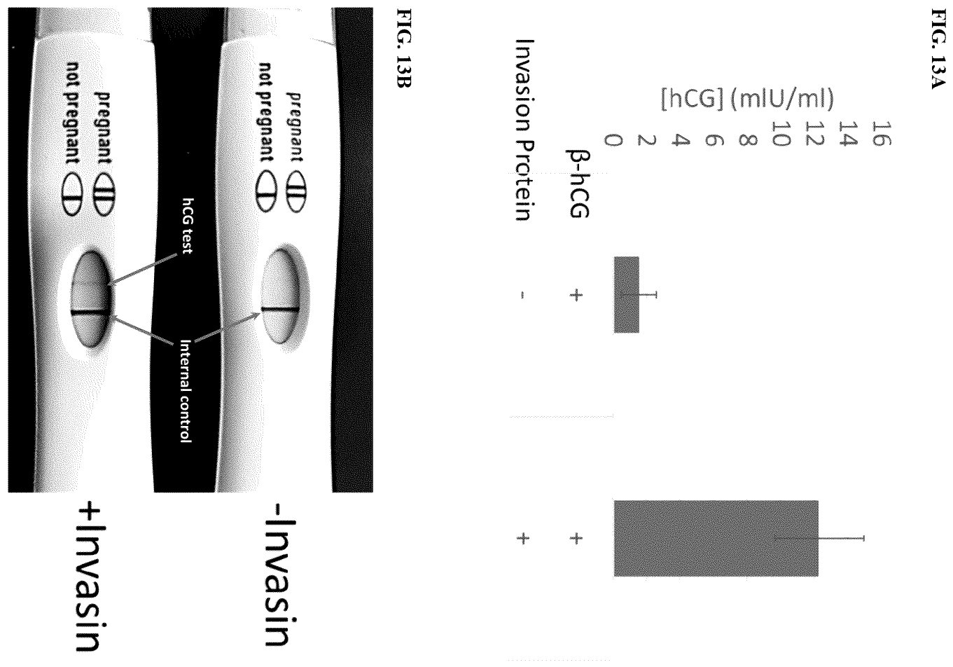

An invasin⁺, listeriolysin⁺, β-hCG⁺E. coli Nissle 1917 dapAΔ strain having the invasin gene a low copy plasmid derived from pMUT1 and a high copy plasmid carrying harboring the human β-chorionic gonadotropin (β-hCG) gene containing an artificial intron under the control of a mammalian promoter (SEQ ID NO: 6) and listeriolysin O (Lysin; SEQ ID NO: 2) under the control of a bacterial promoter, and a selection marker was constructed. An invasin⁻, listeriolysin⁺, β-hCG⁺E. coli Nissle 1917 dapAΔ control strain (lacking invasin gene but carrying the plasmid genes encoding the β-hCG and lysin) was also constructed. To detect the cancer cells, the invasin⁺, listeriolysin⁺, β-hCG⁺ experimental bacteria and the invasin⁻, listeriolysin⁺, β-hCG⁺ control bacteria were seeded onto SW480 cells at identical multiplicities of infection. After 3 hours incubation, cells were washed with PBS and antibiotics were added to kill any extracellular bacteria. Cells were incubated for additional 3 days and samples of media were removed for the measurement of β-hCG. Human β-chorionic gonadotropin (β-hCG) was measured using an ELISA-based assay (β-hCG Elisa RUO kit from DRG International). As shown in FIG. 13A, the cells contacted with the invasin⁺, listeriolysin⁺, β-hCG⁺ strain but not the invasin, listeriolysin⁺, 0-hCG⁺ strain produced β-chorionic gonadotropin in the culture supernatant, as shown by the ELISA test. Culture supernatants were also assayed using an over-the-counter pregnancy test. Summarily, wicks of the pregnancy tests were dipped into media of the SW480 cells treated with the invasin⁺, listeriolysin⁺, β-hCG⁺ strain or the invasin, listeriolysin⁺, β-hCG⁺ strain. As shown in FIG. 13B, the cells contacted with the invasin⁺, listeriolysin⁺, β-hCG⁺ strain but not the invasin⁻, listeriolysin⁺, β-hCG⁺ strain showed the appearance of a line at first “pregnant”-specific location, indicating the production of β-chorionic gonadotropin in the culture supernatant. As expected, the internal control showed a positive response shown by the appearance of a line in both tests (FIG. 13B).

These results demonstrate that the engineered bacteria disclosed herein delivered a DNA payload to cancer cells, which resulted in production and secretion of β-hCG by the cancer cells and thereby facilitated the detection the cancer cells.

### Example 5. In Vivo Detection of Colorectal Carcinoma Cells Using Gaussia Luciferase (Gluc) and Human β-Chorionic Gonadotropin (hCG) Detection Markers

These experiments will be carried out in a mouse model of colorectal cancer. Briefly, normal or Apc^{Fl/Fl}; Vil-Cre-ERT2 mice will be administered 30 μM 4-OH tamoxifen to induce carcinogenesis. Bacteria of the invasin⁺, listeriolysin⁺, Gluc⁺ strain or the invasin⁻, listeriolysin⁺, Gluc⁺ strain will be administered to the mice using an enema and/or gavage. Blood and urine will be recovered from some mice, and Glue will be detected using ELISA or activity assays. Some mice will be sacrificed, colons will be excised, washed and Glue will be detected by immunohistochemistry or luminometry.

It is anticipated that normal mice will not show Gluc expression irrespective of whether they are administered with the invasin⁺, listeriolysin⁺, β-Gluc⁺ strain or the invasin⁻, listeriolysin⁺, β-Gluc⁺ strain. On the other hand, Apc^{Fl/Fl}; Vil-Cre-ERT2 mice are anticipated to show j-Gluc in blood, urine and/or in colons.

In another set of experiments, Apc^{Fl/Fl}; Vil-Cre-ERT2 mice will be administered 30 M 4-OH tamoxifen to induce carcinogenesis. Bacteria of the invasin⁺, listeriolysin⁺, β-hCG⁺ strain or the invasin⁻, listeriolysin⁺, β-hCG⁺ strain will be administered to the mice using an enema and/or gavage. Blood and urine will be recovered from some mice, and β-hCG will be detected using ELISA and/or pregnancy tests. Some mice will be sacrificed, colons will be excised, washed and β-hCG will be detected by immunohistochemistry.

It is anticipated that normal mice will not show β-hCG expression irrespective of whether they are administered with the invasin⁺, listeriolysin⁺, β-hCG⁺ strain or the invasin⁻, listeriolysin⁺, β-hCG⁺ strain. On the other hand, Apc^{Fl/Fl}; Vil-Cre-ERT2 mice are anticipated to show β-hCG in blood, urine and/or in colons.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Sequences of Surface Proteins (Invasin and Analogs)

SEQ ID NO: 1
Amino acid sequence of invasin
MVFQPISEFLLIRNAGMSMYENKIISFNIISRIVICIFLICGMFMAGASEKYDANAPQQVQPYS
VSSSAFENLHPNNEMESSINPFSASDTERNAAIIDRANKEQETEAVNKMISTGARLAASGRASD
VAHSMVGDAVNQEIKQWLNRFGTAQVNLNFDKNESLKESSLDWLAPWYDSASFLFFSQLGIRNK
DSRNTLNLGVGIRTLENGWLYGLNTFYDNDLTGHNHRIGLGAEAWTDYLQLAANGYFRLNGWHS
SRDFSDYKERPATGGDLRANAYLPALPQLGGKLMYEQYTGERVALFGKDNLQRNPYAVTAGINY
TPVPLLTVGVDQRMGKSSKHETQWNLQMNYRLGESFQSQLSPSAVAGTRLLAESRYNLVDRNNN
IVLEYQKQQVVKLTLSPATISGLPGQVYQVNAQVQGASAVREIVWSDAELIAAGGTLTPLSTTQ
FNLVLPPYKRTAQVSRVTDDLTANFYSLSALAVDHQGNRSNSFTLSVTVQQPQLTLTAAVIGDG
APANGKTAITVEFTVADFEGKPLAGQEVVITTNNGALPNKITEKTDANGVARIALTNTTDGVTV
VTAEVEGQRQSVDTHFVKGTIAADKSTLAAVPTSIIADGLMASTITLELKDTYGDPQAGANVAF
DTTLGNMGVITDHNDGTYSAPLTSTTLGVATVTVKVDGAAFSVPSVTVNFTADPIPDAGRSSFT
VSTPDILADGTMSSTLSFVPVDKNGHFISGMQGLSFTQNGVPVSISPITEQPDSYTATVVGNTA
GDVTITPQVDTLILSTLQKKISLFPVPTLTGILVNGQNFATDKGFPKTIFKNATFQLQMDNDVA
NNTQYEWSSSFTPNVSVNDQGQVTITYQTYSEVAVTAKSKKFPSYSVSYRFYPNRWIYDGGTSL
VSSLEASRQCQGSDMSAVLESSRATNGTRAPDGTLWGEWGSLTAYSSDWQSGEYWVKKTSTDFE
TMNMDTGALVQGPAYLAFPLCALAI
SEQ ID NO: 7
Invasin Chromosomal Configuration (Bold: Coding Region, Italics: ProD promoter)
cacagctaacaccacgtcgtccctatctgctgccctaggtctatgagtggttgctggataactt
tacgggcatgcataaggctcgtataatatattcagggagaccacaacggtttccctctacaaat
aattttgtttaactttactagttgtgtaggctggagctgcttcgaagttcctatactttctaga
gaataggaacttcggaataggaactaaggaggatattcatatgATgaattcaggagggtcgaca
tggtctttcagccgatatccgagttcttactgatacgtaacgccggtatgtccatgtactttaa
caaaattatatcgttcaacattatctcaagaattgtcatttgtattttcttgatttgcgggatg
ttcatggctggcgcctcggagaaatacgacgctaatgcgcctcagcaggtgcaaccctattcag
tttcttcgagtgcctttgagaacttgcaccctaataatgaaatggaaagtagtataaatccatt
ctcggcctcagacactgaacgcaacgccgcgataattgaccgcgctaacaaagaacaagaaacg
gaagccgtcaataagatgatctccacaggggctagattagccgcgagcggacgggcttcagatg
ttgcgcactctatggttggtgacgcggtgaatcaggagatcaagcaatggttgaatagattcgg
tactgctcaggtgaacttaaactttgacaaaaacttcagccttaaagagtcttcattagactgg
ttagccccatggtacgactcagcatcctttttattttttagccaattgggcatcagaaataaag
actctcgcaacactctgaaccttggagttggaatacgcaccttagagaacggttggctgtatgg
tttgaatactttctacgataatgacctgactggacacaaccatcgtataggtcttggggcggaa
gcatggacagactacttacagcttgccgcaaatgggtactttagattaaacggctggcatagta
gccgtgatttctctgattataaggagagacctgctacgggaggagatttaagagctaacgcata
cttaccagctttaccacagttaggaggcaaactgatgtatgagcagtacactggggagcgggtc
gccctttttgggaaggataatctgcagcgcaacccttatgcggtcacggctgggattaactata
ctcccgtcccgttattgaccgtgggcgtagatcagcgtatgggtaagagttccaagcatgaaac
gcaatggaatttgcagatgaactaccgtcttggtgaatccttccagagtcagcttagccccagc
gcggtcgctggtactcgcctgttagcagaaagtcggtacaatttagtagatagaaacaacaaca
tagtcttagagtatcagaaacaacaggttgttaagcttacattgtctcccgccaccataagcgg
gctgcctggacaagtctatcaagttaacgcccaggtgcaaggtgcgagcgccgttcgggaaata
gtgtggagcgatgcggaacttatcgccgcgggaggaacgttaacgcctttatccacaacccagt
ttaacttagtgttgcccccctacaagagaaccgcgcaggtatcgcgcgtaaccgacgatttaac
agctaacttctacagtctttccgcattagccgtggaccaccaaggaaacagaagtaactccttc
actcttagcgtcactgttcaacagccacaactgactctgacggctgcggtaattggtgatgggg
cgccagccaatgggaagactgcaattacggttgagttcaccgtagcggatttcgaggggaagcc
acttgcagggcaagaagtagtaataacaactaacaacggtgctcttcctaacaaaattacagaa
aagacggacgcaaacggtgttgctcgcatcgcattaacgaataccactgatggagtaaccgtgg
tcactgcagaagttgaggggcaacgccagtcggtggatactcatttcgtcaaggggacgatagc
agcagacaaatctacgcttgcggccgtgcctaccagcatcatcgcggacggactgatggcatcg
accattacccttgagttgaaggacacctacggcgatcctcaggcgggtgctaatgtggcattcg
atactacactgggcaacatgggagtaataactgaccataacgatggtacttattccgcaccctt
aacaagtactacgttgggtgtagctaccgtaaccgttaaagtcgaTggagccgctttttcggtt
ccgtcggtcaccgtcaatttcacagctgatccgataccagatgcgggccgcagcagcttcaccg
tcagtactccggacatattagctgacgggactatgtcttctacgttgtcctttgtacccgtcga
taagaacggtcattttattagcgggatgcagggtttgtcatttacgcagaatggcgtccctgta
tcaatctcgcctattacggagcaacccgattcctacacagcaacggtagttggaaatacggcag
gtgatgtaacaataacaccccaagttgacacactgatcctgtcaaccttgcagaaaaagatctc
gttgtttcctgttccgaccttaacgggaatcttagtaaatggtcagaattttgccactgacaag
gggttccctaagaccatcttcaaaaatgctacattccagttacagatggacaatgacgtggcca
acaacactcaatacgaatggtcctccagcttcactcctaatgtatcggttaatgaccaaggaca
ggtaaccattacctaccaaacctacagcgaggtcgccgtaactgcgaaatcaaaaaagtttcca
tcctatagcgtatcttacagattctatccgaacagatggatttacgatggaggcacaagtctgg
taagctcgctggaagcatcacggcaatgccaaggtagtgatatgagcgccgtcctggaaagctc
gagagctacaaatggaaccagagctccagatgggactttgtggggtgagtggggcagtttgacc
gcgtacagttcggattggcagtctggggaatattgggtaaaaaagacttccacagatttcgaaa
cgatgaatatggataccggggcgcttgtccaaggtcccgcatatttggcgttcccgctttgcgc
tttagccatctag
Sequences of Lysins (e.g., Listeriolysin O and Derivatives)
SEQ ID NO: 2
Amino acid sequence of listeriolysin O
MKKIMLVFITLILISLPIAQQTEAKDASAFHKEDLISSMAPPTSPPASPKTPIEKKHADEIDKY
IQGLDYNKNNVLVYHGDAVTNVPPRKGYKDGNEYIVVEKKKKSINQNNADIQVVNAISSLTYPG
ALVKANSELVENQPDVLPVKRDSLTLSIDLPGMTNQDNKIVVKNATKSNVNNAVNTLVERWNEK
YAQAYPNVSAKIDYDDEMAYSESQLIAKFGTAFKAVNNSLNVNFGAISEGKMQEEVISFKQIYY
NVNVNEPTRPSRFFGKAVTKEQLQALGVNAENPPAYISSVAYGRQVYLKLSTNSHSTKVKAAFD
AAVSGKSVSGDVELTNIIKNSSFKAVIYGGSAKDEVQIIDGNLGDLRDILKKGATFNRETPGVP
IAYTTNFLKDNELAVIKNNSEYIETTSKAYTDGKINIDHSGGYVAQFNISWDEINYDPEGNEIV
QHKNWSENNKSKLAHFTSSIYLPGNARNINVYAKECTGLAWEWWRTVIDDRNLPLVKNRNISIW
GTTLYPKYSNSVDNPIE
SEQ ID NO: 8
Amino acid sequence of Listeriolysin O lacking periplasmic secretion signal
MDASAFHKEDLISSMAPPTSPPASPKTPIEKKHADEIDKYIQGLDYNKNNVLVYHGDAVINVPP
RKGYKDGNEYIVVEKKKKSINQNNADIQVVNAISSLTYPGALVKANSELVENQPDVLPVKRDSL
TLSIDLPGMTNQDNKIVVKNATKSNVNNAVNTLVERWNEKYAQAYPNVSAKIDYDDEMAYSESQ
LIAKFGTAFKAVNNSLNVNFGAISEGKMQEEVISFKQIYYNVNVNEPTRPSRFFGKAVTKEQLQ
ALGVNAENPPAYISSVAYGRQVYLKLSTNSHSTKVKAAFDAAVSGKSVSGDVELTNIIKNSSFK
AVIYGGSAKDEVQIIDGNLGDLRDILKKGATENRETPGVPIAYTTNFLKDNELAVIKNNSEYIE
TTSKAYTDGKINIDHSGGYVAQFNISWDEINYDPEGNEIVQHKNWSENNKSKLAHFTSSIYLPG
NARNINVYAKECTGLAWEWWRTVIDDRNLPLVKNRNISIWGTTLYPKYSNSVDNPIE
Sequences of illustrative selection markers
SEQ ID NO: 5
Alanine Racemase with promoter (Bold: Coding Region, Italics: promoter region)
ccgtcgcggcaggtgttatacctgcgattagcagaaaaaagaaaacaaattttatctttgtcat
ggctgaaaatccgatatcatcttgccgtttataaacccatccctggatgtggaatagcatactc
cggtcatatctgtttagcgaggttttgcactggtgggcagaacctgaaaaaaaggaatacaaat
gcaagcggcaactgttttgattaaccgccgcgctctgcgacacaacctgcaacgtctgcgtgaa
ctggcccccgccagtaaactggttgcggtggtgaaagcgaacgcctacggtcacggtctgattg
agaccgcgcgaacgctccccgatgctgacgcctttggcgttgcccgtctcgaagaagccctacg
gctgcgggcggggggtatcacgcgacctattttgttactggaaggtttttttgaagcagacgat
ttgccgacgatctccgctgaacatctgcataccgcagtccacaatgaagagcagcttgtcgccc
tcgaaaacgctgaacttaaagagcctgtcaccgtctggatgaagctcgataccggaatgcaccg
tttgggcgtattgccggaacaggccgaggcgttttatcagcgtctgagccagtgtaaaaatgtc
cgccagccggtgaacattgttagtcacttcgcccgtgccgatgaaccgcaaagcggcgcgactg
aaaagcagctcgatatcttcaacaccttttgtgaaggtaaaccggggcaacgctcaattgcggc
atcaggcggcattttgttatggccgcagtcgcattttgactgggcgcgtccggggatcattctt
tacggtgtctcgccgctggaagatggcacgacaggggctgattttggctgtcagccagtcatgt
ctttaacctccagcctgattgccgtgcgtgagcataaagccggagagcctgtcggttatggtgg
aacctgggtaagcgaacgtgatactcgtcttggcgtagtcgcgatgggctatggcgatggttat
ccgcgcgccgcgccgtccggtacgccagtgctggtgaacggtcgcgaagtgccgattgtcgggc
gagtcgcgatggatatgatctgcgtagacttaggtccacaggcgcaggacaaagccggggaccc
ggtcattttatggggcgaaggtttgcccgtagaacgtatcgctgaaatgacgaaagtaagcgct
tacgaacttattacgcgcctgacttcaagagtcgcgatgaaatacgtggattaa
Sequences of illustrative secretable biomarkers
SEQ ID NO: 3
Gaussia Luciferase Construct (Bold: Coding Region, Italics: CMV promoter)
Agtaatcaattacggggtcattagttcatagcccatatatggagttccgcgttacataacttac
ggtaaatggcccgcctggctgaccgcccaacgacccccgcccattgacgtcaataatgacgtat
gttcccatagtaacgccaatagggactttccattgacgtcaatgggtggagtatttacggtaaa
ctgcccacttggcagtacatcaagtgtatcatatgccaagtacgccccctattgacgtcaatga
cggtaaatggcccgcctggcattatgcccagtacatgaccttatgggactttcctacttggcag
tacatctacgtattagtcatcgctattaccatgctgatgcggttttggcagtacatcaatgggc
gtggatagcggtttgactcacggggatttccaagtctccaccccattgacgtcaatgggagttt
gttttggcaccaaaatcaacgggactttccaaaatgtcgtaacaactccgccccattgacgcaa
atgggcggtaggcgtgtacggtgggaggtctatataagcagagctggtttagtgaaccgtcaga
tcagatctttgtcgatcctaccatccactcgacacacccgccagcggccgctgccaagcttggt
accgccaccATGGGAGTCAAAGTTCTGTTTGCCCTGATCTGCATCGCTGTGGCCGAGGCCAAGC
CCACCGAGAACAACGAAGACTTCAACATCGTGGCCGTGGCCAGCAACTTCGCGACCACGGATCT
CGATGCTGACCGCGGGAAGTTGCCCGGCAAGAAGCTGCCGCTGGAGGTGCTCAAAGAGTTGGAA
GCCAATGCCCGGAAAGCTGGCTGCACCAGGGGCTGTCTGATCTGCCTGTCCCACATCAAGTGCA
CGCCCAAGATGAAGAAGTTCATCCCAGGACGCTGCCACACCTACGAAGGCGACAAAGAGTCCGC
ACAGGGCGGCATAGGCGAGGCGATCGTCGACATTCCTGAGATTCCTGGGTTCAAGGACTTGGAG
CCCTTGGAGCAGTTCATCGCACAGGTCGATCTGTGTGTGGACTGCACAACTGGCTGCCTCAAAG
GGCTTGCCAACGTGCAGTGTTCTGACCTGCTCAAGAAGTGGCTGCCGCAACGCTGTGCGACCTT
TGCCAGCAAGATCCAGGGCCAGGTGGACAAGATCAAGGGGGCCGGTGGTGACTAA
SEQ ID NO: 4
Gaussia Luciferase-Intron Construct (Bold: Coding Region, Italics: CMV promoter,
Underline: Intron)
agtaatcaattacggggtcattagttcatagcccatatatggagttccgcgttacataacttac
ggtaaatggcccgcctggctgaccgcccaacgacccccgcccattgacgtcaataatgacgtat
gttcccatagtaacgccaatagggactttccattgacgtcaatgggtggagtatttacggtaaa
ctgcccacttggcagtacatcaagtgtatcatatgccaagtacgccccctattgacgtcaatga
cggtaaatggcccgcctggcattatgcccagtacatgaccttatgggactttcctacttggcag
tacatctacgtattagtcatcgctattaccatgctgatgcggttttggcagtacatcaatgggc
gtggatagcggtttgactcacggggatttccaagtctccaccccattgacgtcaatgggagttt
gttttggcaccaaaatcaacgggactttccaaaatgtcgtaacaactccgccccattgacgcaa
atgggcggtaggcgtgtacggtgggaggtctatataagcagagctggtttagtgaaccgtcaga
tcagatctttgtcgatcctaccatccactcgacacacccgccagcggccgctgccaagcttggt
accgccaccATGGGAGTCAAAGTTCTGTTTGCCCTGATCTGCATCGCTGTGGCCGAGGCCAAGC
CCACCGAGAACAACGAAGACTTCAACATCGTGGCCGTGGCCAGCAACTTCGCGACCACGGATCT
CGATGCTGACCGCGGGAAGTTGCCCGGCAAGAAGCTGCCGCTGGAGGTGCTCAAAGAGTTGGAA
GCCAATGCCCGGAAAGCTGGCTGCACCAGGGGCTGTCTGATCTGCCTGTCCCACATCAAGTGCA
CGCCCAAGATGAAGAAGTTCATCCCAGGACGCTGCCACACCTACGAAGGCGAgtaagtcgactc
gttggatccccactacagccgatactcaagcttgacgaattcgagtatccaaggtagtggacta
gtgtgacgctgctgacccctttctttcccttctgcagCAAAGAGTCCGCACAGGGCGGCATAGG
CGAGGCGATCGTCGACATTCCTGAGATTCCTGGGTTCAAGGACTTGGAGCCCTTGGAGCAGTTC
ATCGCACAGGTCGATCTGTGTGTGGACTGCACAACTGGCTGCCTCAAAGGGCTTGCCAACGTGC
AGTGTTCTGACCTGCTCAAGAAGTGGCTGCCGCAACGCTGTGCGACCTTTGCCAGCAAGATCCA
GGGCCAGGTGGACAAGATCAAGGGGGCCGGTGGTGACTAA
SEQ ID NO: 6
β-hCG Construct (Bold: Coding Region, Italics: CMV promoter, Underline: Intron)
agtaatcaattacggggtcattagttcatagcccatatatggagttccgcgttacataacttac
ggtaaatggcccgcctggctgaccgcccaacgacccccgcccattgacgtcaataatgacgtat
gttcccatagtaacgccaatagggactttccattgacgtcaatgggtggagtatttacggtaaa
ctgcccacttggcagtacatcaagtgtatcatatgccaagtacgccccctattgacgtcaatga
cggtaaatggcccgcctggcattatgcccagtacatgaccttatgggactttcctacttggcag
tacatctacgtattagtcatcgctattaccatgctgatgcggttttggcagtacatcaatgggc
gtggatagcggtttgactcacggggatttccaagtctccaccccattgacgtcaatgggagttt
gttttggcaccaaaatcaacgggactttccaaaatgtcgtaacaactccgccccattgacgcaa
atgggcggtaggcgtgtacggtgggaggtctatataagcagagctggtttagtgaaccgtcaga
tcagatctttgtcgatcctaccatccactcgacacacccgccagcggccgctgccaagcttggt
accgccaccATGGAGATGTTCCAGGGACTGCTGTTACTTCTTCTTTTGAGCATGGGAGGTACGT
GGGCTTCTAAGGAGCCATTACGTCCACGCTGCCGTCCTATTAACGCCACCTTGGCCGTAGAGAA
GGAAGGCTGTCCCGTCTGTATTACAGTGAACACTACAATCTGCGCTGGGTATTGTCCAACTATG
ACCCGTGTCTTGCAAGGGGTTCTTCCCGCCTgtaagtcgactcgttggatccccactacagccg
atactcaagcttgacgaattcgagtatccaaggtagtggactagtgtgacgctgctgacccctt
tctttcccttctgcagTACCACAAGTAGTCTGCAATTACCGCGATGTCCGTTTTGAAAGTATCC
GTTTGCCGGGATGTCCGCGCGGAGTGAACCCGGTGGTGTCCTACGCGGTTGCACTGAGCTGTCA
GTGCGCTTTATGTCGCCGCAGTACCACGGATTGCGGAGGGCCCAAGGATCACCCTTTGACCTGT
GATGATCCACGTTTCCAGGACAGTAGTAGCTCAAAAGCACCTCCTCCGAGCCTTCCAAGCCCCT
CCCGTCTGCCAGGCCCGTCGGATACCCCCATTTTACCACAGTAA

I/We claim:

1. A method for detecting diseased epithelial tissue, and optionally gastrointestinal (GI) tissue, comprising:
(a) administering to the epithelial tissue of a subject, an engineered microorganism that specifically invades diseased cells of the epithelium, and directs expression of a secretable biomarker in the diseased cells;
(b) obtaining a biological sample of the subject; and
(c) evaluating the sample for the presence or amount of the secretable biomarker.

2. The method of claim 1, wherein the genetically engineered microorganism comprises an exogenous gene encoding a surface protein that specifically interacts with one or more cell membrane receptor(s) that are specifically exposed on the luminal side of epithelial cells of diseased tissue; and
wherein the surface protein promotes binding and/or invasion of the microorganism in the diseased epithelial cells.

3. The method of claim 1 or claim 2, wherein the biological sample is selected from blood, plasma, serum, urine, feces, saliva, and mucus, or a combination of any two or more thereof.

4. The method of claim 3, wherein the secretable biomarker is excreted in a biological fluid selected from mucus, saliva and urine.

5. The method of any one of claims 1-4, wherein the genetically engineered microorganism is non-pathogenic.

6. The method of claim 5, wherein the genetically engineered microorganism harbors at least one auxotrophic mutation.

7. The method of any one of claims 1-6, wherein the secretable biomarker is expressed from a mammalian promoter.

8. The method of claim 7, wherein the mammalian promoter directs GI tract epithelial cell-specific expression.

9. The method of any one of claims 1-6, wherein the secretable biomarker is expressed from a microbial promoter, and the mRNA encoding the secretable biomarker is translated in the diseased cells.

10. The method of claim 9, wherein the RNA encoding the secretable biomarker further comprises an internal ribosome entry site (IRES).

11. The method of any one of claims 1-10, wherein the genetically engineered microorganism is administered via oral or rectal route.

12. The method of any one of claims 1 to 11, wherein the secretable biomarker is an enzyme, a peptide hormone, or a protein or a peptide antigen.

13. The method of claim 12, wherein the secretable biomarker is not normally present in the biological fluid of the subject.

14. The method of claim 12 or claim 13, wherein the secretable biomarker is a secreted protein selected from alkaline phosphatase, a human chorionic gonadotropin, a human carcinoembryonic antigen (CEA), colon cancer secreted protein 2, cathepsin B, a Gaussia luciferase (Gluc), a Metridia luciferase (MLuc), a subunit thereof, a fragment thereof, and a combination of any two or more thereof.

15. The method of claim 14, wherein the secretable biomarker is optimized for distribution in body fluids (e.g. increased excretion in the urine, or increased accumulation in the blood).

16. The method of any one of claims 1 to 15, wherein the secretable biomarker is measured qualitatively, quantitatively or semi-quantitatively, optionally wherein the secretable biomarker is measured without the use of a clinical laboratory instrument selected from a colonoscope, an endoscope, a radiography instrument, a computed tomography (CT) scanner, a magnetic resonance imaging (MRI) machine, a blood gas analyzer, and an urine chemistry analyzer.

17. The method of claim 15, wherein the secretable biomarker is measured by agglutination, amperometry, atomic absorption spectrometry, atomic emission spectrometry, circular dichroism (CD), chemiluminescence, colorimetry, dipstick assay, lateral flow immunoassay, fluorimetry, electrophoretic assay, enzymatic assay, enzyme linked immunosorbant assay (ELISA), gas chromatography, gravimetry, high pressure liquid chromatography (HPLC), immunoassay, immunofluorometric enzyme assay, mass spectrometry, nuclear magnetic resonance, radioimmunoassay, spectrometry, titrimetry, western blotting, or a combination of any two or more thereof.

18. The method of claim 17, wherein the secretable biomarker is measured by an assay selected from lateral flow or dipstick immunoassay or enzymatic assay.

19. The method of any one of claims 15-18, wherein the secretable biomarker is measured using a portable and/or handheld instrument.

20. The method of claim 19, wherein measuring of the secretable biomarker yields a numerical value.

21. The method of any one of claims 14 to 20, wherein the secretable biomarker comprises a secreted alkaline phosphatase, optionally wherein the secreted alkaline phosphatase is a truncated form of human placental alkaline phosphatase.

22. The method of claim 21, wherein the secreted alkaline phosphatase is measured using an enzymatic assay.

23. The method of claim 22, wherein the secreted alkaline phosphatase is measured using a colorimetric, fluorimetric and/or chemiluminiscent readout.

24. The method of claim 23, wherein the assay uses a substrate selected from p-nitrophenyl phosphate (pNPP), 4-Methylumbelliferyl phosphate disodium salt (MUP), and Chloro-5-substituted adamantyl-1,2-dioxetane phosphate (CSPD).

25. The method of any one of claims 14 to 20, wherein the secretable biomarker comprises a human chorionic gonadotropin (hCG), or a subunit thereof, or a fragment thereof.

26. The method of claim 25, wherein the hCG, or the subunit thereof, or the fragment thereof is measured using an immunoassay.

27. The method of claim 26, wherein the hCG, or the subunit thereof, or the fragment thereof is measured using an anti-hCG antibody, or a fragment thereof.

28. The method of claim 27, wherein the immunoassay is selected from an agglutination assay, a dipstick assay, a lateral flow assay, a quantitative immunoassay, enzyme linked immunosorbant assay (ELISA), or a combination of any two or more thereof.

29. The method of claim 28, wherein the hCG, or the subunit thereof, or the fragment thereof is measured using chemiluminescence, colorimetry, and/or fluorescence.

30. The method of any one of claims 25-29, wherein the biological sample is selected from blood, urine, and saliva.

31. The method of any one of claims 14 to 20, wherein the secretable biomarker comprises a luciferase.

32. The method of claim 31, wherein the luciferase is Gaussia luciferase (Gluc) or a derivative thereof.

33. The method of claim 31 or claim 32, wherein luciferase is measured using an enzymatic assay.

34. The method of claim 33, wherein the enzymatic assay uses a luciferin as a substrate.

35. The method of claim 34, wherein the luciferin is coelenterazine.

36. The method of any one of claims 31-35, wherein the luciferase is measured using a luminescence readout.

37. The method of any one of claims 31-36, wherein the biological sample is selected from blood, urine, and saliva.

38. The method of any one of claims 1 to 37, wherein a gene encoding the secretable biomarker comprises at least one intron.

39. The method of claim 38, wherein the at least one intron is a spliceosomal intron.

40. The method of any one of claims 1 to 39, wherein the microorganism is selected from Lactobacillus, Bifidobacterium, Saccharomyces, Enterococcus, Streptococcus, Pediococcus, Leuconostoc, Bacillus, Escherichia coli.

41. The method of claim 40, wherein the microorganism is Escherichia coli, and optionally Escherichia coli Nissle 1917 or a derivative thereof.

42. The method of claim 41, wherein the Escherichia coli Nissle 1917 or the derivative thereof harbors a plasmid pMUT1 and/or a plasmid pMUT2, and/or derivatives thereof.

43. The method of claim 41, wherein the Escherichia coli Nissle 1917 or the derivative thereof is cured of the plasmid pMUT1 and/or the plasmid pMUT2.

44. The method of any one of claims 2 to 43, wherein the surface protein specifically interacts with one or more cell membrane receptor(s) that are not exposed on the luminal side of normal epithelial cells of gastrointestinal tissue.

45. The method of claim 44, wherein the surface protein is an invasin, or a fragment thereof.

46. The method of claim 45, wherein the invasin is selected from Yersinia enterocolitica invasin, Yersinia pseudotuberculosis invasin, Salmonella enterica PagN, Candida albicans Als3 and E. coli intimin.

47. The method of claim 44, wherein the surface protein comprises a peptide or protein that specifically binds to the surface of cancerous cells and/or pre-cancerous cells.

48. The method of claim 47, wherein the peptide or protein is selected from a leptin, an antibody, or a fragment thereof.

49. The method of claim 48, wherein the antibody, or a fragment thereof is selected from a single-domain antibody (sdAb) and an scFv fragment.

50. The method of any one of claims 47-49, wherein the peptide or protein is displayed on a microbial surface protein.

51. The method of claim 50, wherein the peptide or protein is expressed as a fusion protein with the microbial surface protein.

52. The method of claim 51, wherein the microbial surface protein is selected from invasin, intimin and adhesin.

53. The method of any one of claims 1 to 52, wherein the microorganism further comprises an exogenous gene encoding a lysin that is capable of lysing the endocytotic vacuole.

54. The method of claim 53, wherein the lysin is listeriolysin O, or a mutant derivative thereof.

55. The method of claim 53 or 54, wherein the gene encoding the surface protein and/or the gene encoding the lysin is integrated in the genome of the microorganism.

56. The method of claim 55, wherein the gene encoding the surface protein, and the gene encoding the lysin are integrated at a single genomic site.

57. The method of claim 56, wherein the single genomic site is selected from an integration site of a bacteriophage and an integration site of a plasmid.

58. The method of claim 53 or 54, wherein the gene encoding the surface protein, and/or the gene encoding the lysin are inserted on a plasmid.

59. The method of any one of claims 1 to 58, wherein the gene encoding the secretable biomarker is integrated in a genome of the microorganism or inserted on a plasmid.

60. The method of claim 59, the gene encoding the secretable biomarker is integrated at the single genomic site.

61. The method of claim 59, wherein the gene encoding the secretable biomarker is inserted on a plasmid.

62. The method of any one of claims 1 to 57, wherein the gene encoding the secretable biomarker is inserted on a second plasmid.

63. The method of claim 62, wherein the microorganism is Escherichia coli Nissle 1917 or a derivative thereof and the plasmid and/or the second plasmid is selected from the plasmid pMUT1, the plasmid pMUT2, and a derivative thereof.

64. The method of claim 62 or claim 63, wherein the plasmid and/or the second plasmid comprises a selection mechanism.

65. The method of claim 64, wherein the selection mechanism is selected from an antibiotic resistance marker, a toxin-antitoxin system, a marker causing complementation of a mutation in an essential gene, a cis acting genetic element and a combination of any two or more thereof.

66. The method of claim 64, wherein the selection mechanism is an antibiotic resistance marker selected from kanamycin resistance gene and tetracycline resistance gene.

67. The method of claim 64, wherein the selection mechanism is a toxin-antitoxin system selected from a hok/sok system of plasmid R1, parDE system of plasmid RK2, ccdAB of F plasmid, flmAB of F plasmid, kis/kid system of plasmid R1, XCV2162-ptaRNA1 of Xanthomonas campestris, ataT-ataR of enterohemorragic E. coli or Klebsiella, toxIN system of Erwinia carotovora, parE-parD system of Caulobacter crescentus, fst-RNAII from Enterococcus faecalis plasmid AD1, ε-ζ system of Bacillus subtilis plasmid pSM19035 and a combination of any two or more thereof.

68. The method of claim 64, wherein the selection mechanism is a marker causing complementation of a mutation in an essential gene, where the essential gene product encodes:
an enzyme involved in biosynthesis of an essential nutrient or a cell wall component; and/or
an house-keeping function.

69. The method of claim 68, wherein the essential gene is selected from dapA, dapD, murA, alr, dadX, murI, dapE, thyA and a combination of any two or more thereof, optionally wherein the essential genes are alr and dadX, optionally wherein the genetically engineered microorganism has an inactivation of alr and dadX, and the selection mechanism is plasmid having an alr gene to complement the alr and dadX inactivation.

70. The method of claim 68, wherein the house-keeping function is selected from an rRNA, a tRNA, infA, a gene encoding a subunit of an RNA polymerase, a DNA polymerase, a cell division protein, or a chaperon protein, and a combination of any two or more thereof.

71. The method of claim 64, wherein the selection mechanism is a cis acting genetic element selected from a ColE1 cer locus and pSC101 par locus.

72. The method of any one of claims 1-64, wherein the microorganism harbors at least one mutation selected from a deletion, inactivation, or altered expression or activity of one or more of dapA, dapD, dapE, murA, alr, dadX, murI, thyA, and aroC, and optionally comprises a deletion or inactivation of dapA, alr, and dadX.

73. The method of any one of claims 59 to 72, wherein a plasmid carrying the gene encoding the secretable biomarker comprises at least one binding site for a DNA binding protein.

74. The method of claim 73, wherein at least one binding site for the DNA binding protein is selected from SV40 enhancer, a transcription factor binding site, and a bacterial operators.

75. The method of claim 73, wherein the DNA binding protein comprises one or more nuclear localization signal(s) (NLS).

76. The method of claim 75, wherein the NLS is SV40 T antigen NLS sequence (KKKRKV).

77. The method of any one of any one of claims 73-76, wherein the microorganism comprises a gene encoding the DNA binding protein.

78. The method of any one of claims 73-77, wherein the DNA binding protein is NFκB.

79. The method of any one of claims 1 to 78, wherein the disease is selected from a precancerous lesion, cancer, ulcerative colitis, Crohn's disease, Barrett's esophagus, irritable bowel syndrome and irritable bowel disease.

80. The method of claim 79, wherein the precancerous lesion comprises a polyp selected from sessile polyp, serrated polyp (e.g. hyperplastic polyps, sessile serrated adenomas/polyps, and traditional serrated adenoma), sessile serrated polyp, flat polyp, sub-pedunculated polyp, pedunculated polyp, and a combination thereof.

81. The method of claim 79, wherein the precancerous lesion comprises a biliary intraepithelial neoplasm (BilIN) selected from BilIN-1, BilIN-2, BilIN-3, and cholangiocarcinoma.

82. The method of claim 79, wherein the precancerous lesion comprises a pancreatic intraepithelial neoplasm (PanIN) selected from PanIN-1, PanIN-2, PanIN-3 and pancreatic ductal adenocarcinoma (PDAC).

83. The method of any one of claims 79-82, wherein the precancerous lesion is a diminutive polyp.

84. The method of any one of claims 79-82, wherein the precancerous lesion has a size of from about 0.05 mm to about 30 mm.

85. The method of claim 84, wherein the precancerous lesion has a size of less than about 0.1 mm, less than about 0.25 mm, less than about 0.5 mm, less than about 1 mm, less than about 2 mm, less than about 5 mm, less than about 8 mm, less than about 10 mm, less than about 15 mm, less than about 20 mm, less than about 25 mm, or less than about 30 mm.

86. The method of claim 79, wherein the cancer comprises a polyp, an adenoma, or a frank cancer.

87. The method of claim 79, wherein the cancer comprises Lynch syndrome cancer, familial adenomatous polyposis, hereditary non-polyposis colon cancer (HNPCC), pancreatic ductal adenocarcinoma (PDAC), cholangiocarcinoma, or a sporadic cancer.

88. The method of any one of claims 1 to 79, wherein the subject is predisposed to cancer.

89. The method of claim 88, wherein the subject suffers from a condition selected from Lynch Syndrome, hereditary non-polyposis colon cancer (HNPCC), familial adenomatous polyposis (FAP), Gardner's Syndrome, Turcot's Syndrome, MUTYH-associated polyposis, Peutz-Jeghers syndrome, and juvenile polyposis syndrome and colitis-associated colorectal cancer (CACC).

90. The method of any one of claims 1 to 89, wherein the steps (a), (b) and/or (c) are repeated.

91. The method of claim 90, wherein the steps (a), (b) and/or (c) are repeated no more frequently than, or about monthly, bimonthly, every six months, or annually.

92. A genetically engineered microorganism comprising a gene encoding a surface protein, wherein the surface protein specifically interacts with one or more cell membrane receptor(s) that are specifically exposed on the luminal side of epithelial cells of diseased tissue, and which is optionally epithelial tissue of the gastrointestinal tract,
wherein the surface protein promotes binding and invasion of epithelial cells of diseased tissue,
wherein the microorganism comprises a gene encoding a secretable biomarker that is secretable by the epithelial cells, the gene encoding the secretable biomarker being operably linked to a promoter.

93. The genetically engineered microorganism of claim 92, wherein the genetically engineered microorganism is non-pathogenic.

94. The genetically engineered microorganism of claim 92 or claim 93, wherein the genetically engineered microorganism harbors at least one auxotrophic mutation.

95. The genetically engineered microorganism of any one of claims 92-94, wherein the secretable biomarker is expressed from a mammalian promoter.

96. The genetically engineered microorganism of claim 95, wherein the mammalian promoter directs GI tract epithelial cell-specific expression.

97. The genetically engineered microorganism of any one of claims 92 to 94, wherein the secretable biomarker is expressed from a microbial promoter.

98. The genetically engineered microorganism of claim 97, wherein the RNA encoding the secretable biomarker comprises an internal ribosome entry site (IRES).

99. The genetically engineered microorganism of claim 97 or 98, wherein the microbial promoter is inducible and/or repressible.

100. The genetically engineered microorganism of any one of claims 92-99, wherein the secretable biomarker is an enzyme, peptide hormone, or a protein or peptide antigen.

101. The genetically engineered microorganism of claim 100, wherein the secretable biomarker is not normally present in a biological fluid of the subject selected from blood, urine, or saliva.

102. The genetically engineered microorganism of any one of claims 92-101, wherein the secretable biomarker is selected from alkaline phosphatase, a human chorionic gonadotropin, a human carcinoembryonic antigen (CEA), colon cancer secreted protein 2, Cathepsin B, a Gaussia luciferase (Gluc), a Metridia luciferase (MLuc), a subunit thereof, a fragment thereof, and a combination of any two or more thereof.

103. The genetically engineered microorganism of claim 102, wherein the secretable biomarker is a secreted alkaline phosphatase (SEAP).

104. The genetically engineered microorganism of claim 102, wherein the secretable biomarker is a human chorionic gonadotropin, or a subunit thereof or a fragment thereof.

105. The genetically engineered microorganism of claim 102, wherein the secretable biomarker is a luciferase.

106. The genetically engineered microorganism of claim 105, wherein the secretable biomarker is a luciferase Gaussia luciferase (Gluc) or a derivative thereof.

107. The genetically engineered microorganism of any one of claims 92 to 106, wherein the gene encoding the secretable biomarker comprises at least one intron.

108. The genetically engineered microorganism of any one of claims 92 to 107, wherein the microorganism is selected from Lactobacillus, Bifidobacterium, Saccharomyces, Enterococcus, Streptococcus, Pediococcus, Leuconostoc, Bacillus, Escherichia coli.

109. The genetically engineered microorganism of claim 108, wherein the microorganism is a probiotic Escherichia coli strain or a derivative thereof.

110. The genetically engineered microorganism of claim 109, wherein the microorganism is Escherichia coli Nissle 1917 or a derivative thereof.

111. The genetically engineered microorganism of claim 110, wherein the Escherichia coli Nissle 1917 or the derivative thereof harbors a plasmid pMUT1 and/or a plasmid pMUT2, and/or derivatives thereof.

112. The genetically engineered microorganism of claim 110, wherein the Escherichia coli Nissle 1917 or the derivative thereof is cured of the plasmid pMUT1 and/or the plasmid pMUT2.

113. The genetically engineered microorganism of any one of claims 92 to 112, wherein the surface protein comprises a peptide or protein that specifically binds to the surface of cancerous and/or pre-cancerous cells.

114. The genetically engineered microorganism of claim 113, wherein the surface protein is an invasin, or a fragment thereof.

115. The genetically engineered microorganism of claim 114, wherein the invasin is selected from Yersinia enterocolitica invasin, Yersinia pseudotuberculosis invasin, Salmonella enterica PagN, Candida albicans Als3 and E. coli intimin.

116. The genetically engineered microorganism of claim 113, wherein the peptide or protein is selected from a leptin, an antibody, or a fragment thereof.

117. The genetically engineered microorganism of claim 116, wherein the antibody, or a fragment thereof is selected from a single-domain antibody (sdAb) and an scFv fragment.

118. The genetically engineered microorganism of claim 117, wherein the peptide or protein is displayed on a microbial surface protein.

119. The genetically engineered microorganism of claim 118, wherein the peptide or protein is expressed as a fusion protein with the microbial surface protein.

120. The genetically engineered microorganism of claim 119, wherein the microbial surface protein is selected from invasin, intimin and adhesin.

121. The genetically engineered microorganism of any one of claims 92 to 120, wherein the microorganism further comprises an exogenous gene encoding a lysin that lyses an endocytotic vacuole.

122. The genetically engineered microorganism of claim 121, wherein the lysin is listeriolysin O, or a mutant derivative thereof.

123. The genetically engineered microorganism of claim 121 or claim 122, wherein the gene encoding the surface protein and/or the gene encoding the lysin is integrated in genome of the microorganism.

124. The genetically engineered microorganism of any one of claims 121-123, wherein the gene encoding the surface protein, and the gene encoding the lysin are integrated at a single genomic site.

125. The genetically engineered microorganism of claim 124, wherein the single genomic site is an integration site of a bacteriophage, or an integration site of a plasmid.

126. The genetically engineered microorganism of any one of claims 121-123, wherein the gene encoding the surface protein, and/or the gene encoding the lysin are inserted on a plasmid.

127. The genetically engineered microorganism of claim 126, wherein the gene encoding the secretable biomarker is inserted on the plasmid.

128. The genetically engineered microorganism of any one of claims 92-126, wherein the gene encoding the secretable biomarker is integrated in a genome of the microorganism, optionally, wherein the gene encoding the secretable biomarker are integrated at the single genomic site.

129. The genetically engineered microorganism of any one of claims 92-126, wherein the gene encoding the secretable biomarker is inserted on a second plasmid.

130. The genetically engineered microorganism of any one of claims 125-129, wherein the plasmid and/or the second plasmid comprises a selection mechanism.

131. The genetically engineered microorganism of claim 130, wherein the selection mechanism is selected from an antibiotic resistance marker, a toxin-antitoxin system, a marker causing complementation of a mutation in an essential gene, a cis acting genetic element and a combination of any two or more thereof.

132. The genetically engineered microorganism of claim 130, wherein the selection mechanism is an antibiotic resistance marker selected from kanamycin resistance gene and tetracycline resistance gene.

133. The genetically engineered microorganism of claim 130, wherein the selection mechanism is a toxin-antitoxin system selected from a hok/sok system of plasmid R1, parDE system of plasmid RK2, ccdAB of F plasmid, flmAB of F plasmid, kis/kid system of plasmid R1, XCV2162-ptaRNA1 of Xanthomonas campestris, ataT-ataR of enterohemorragic E. coli or Klebsiella, toxIN system of Erwinia carotovora, parE-parD system of Caulobacter crescentus, fst-RNAII from Enterococcusfaecalis plasmid AD1, ε-ζ system of Bacillus subtilis plasmid pSM19035 and a combination of any two or more thereof.

134. The genetically engineered microorganism of claim 130, wherein the selection mechanism is a marker causing complementation of a mutation in an essential gene, wherein the essential gene encodes
an enzyme involved in biosynthesis of an essential nutrient or a cell wall component; and/or
an house-keeping function.

135. The genetically engineered microorganism of claim 134, wherein the essential gene is selected from dapA, dapD, murA, alr, dadX, murI, dapE, thyA and a combination of any two or more thereof, optionally wherein the essential genes are alr and dadX, optionally wherein the genetically engineered microorganism has an inactivation of alr and dadX, and the selection mechanism is plasmid having an alr gene to complement the alr and dadX inactivation.

136. The genetically engineered microorganism of claim 121, wherein the essential gene encodes a house-keeping function selected from an rRNA, a tRNA, infA, a gene encoding a subunit of an RNA polymerase, a DNA polymerase, a cell division protein, or a chaperon protein, and a combination of any two or more thereof.

137. The genetically engineered microorganism of claim 118, wherein the essential gene is a cis acting genetic element selected from ColE1 cer locus or pSC101 par locus.

138. The genetically engineered microorganism of any one of claims 92-137, wherein the microorganism harbors at least one mutation selected from a deletion, inactivation, or reduced expression or activity of one or more of dapA, dapD, dapE, murA, alr, dadX, murI, thyA, and aroC, wherein the microorganism optionally comprises a deletion or inactivation or dapA, alr, and dadX.

139. The genetically engineered microorganism of any one of claims 129-138, wherein the plasmid or the second plasmid comprises at least one binding site for a DNA binding protein.

140. The genetically engineered microorganism of claim 139, wherein the DNA binding protein comprises one or more nuclear localization signal(s) (NLS).

141. The genetically engineered microorganism of claim 140, wherein the NLS is SV40 T antigen NLS sequence (KKKRKV).

142. The genetically engineered microorganism of any one of any one of claims 139-141, wherein the microorganism comprises a gene encoding the DNA binding protein.

143. The genetically engineered microorganism of any one of claims 139-142, wherein the DNA binding protein is NFκB.

144. The genetically engineered microorganism of any one of claims 139-143, wherein the microorganism comprises a gene encoding the DNA binding protein.

145. A method of diagnosis and/or treatment of a disease in a subject, the method comprising:
(i) administering to the gastrointestinal tract of the subject the genetically engineered microorganism of any one of claims 92 to 144; and
(ii) obtaining a biological sample from the subject; and
(iii) measuring the secretable biomarker in the biological sample to thereby detecting diseased epithelial cells,
optionally wherein the method further comprises administering a treatment to the subject.

146. The method of claim 145 further comprising:
(iv) selecting the subject for treatment if expression of the secretable biomarker is observed.

147. The method of claim 145 or claim 146, wherein the disease is selected from a precancerous lesion, cancer, ulcerative colitis, Crohn's disease, Barrett's esophagus, irritable bowel syndrome and irritable bowel disease.

148. The method of claim 147, wherein the precancerous lesion comprises:
a polyp selected from sessile polyp, serrated polyp, hyperplastic polyps, sessile serrated adenomas/polyps, traditional serrated adenoma, sessile serrated polyp, flat polyp, sub-pedunculated polyp, pedunculated polyp, and a combination thereof;
a biliary intraepithelial neoplasm (BilIN) selected from BilIN-1, BilIN-2, BilIN-3, and cholangiocarcinoma; and/or
a pancreatic intraepithelial neoplasm (PanIN) selected from PanIN-1, PanIN-2, PanIN-3 and pancreatic ductal adenocarcinoma (PDAC).

149. The method of claim 148, wherein the polyp is a diminutive polyp.

150. The method of claim 148, wherein the precancerous lesion has a size of from about 0.05 mm to about 30 mm.

151. The method of claim 150, wherein the precancerous lesion has a size of less than about 0.1 mm, less than about 0.25 mm, less than about 0.5 mm, less than about 1 mm, less than about 2 mm, less than about 5 mm, less than about 8 mm, less than about 10 mm, less than about 15 mm, less than about 20 mm, less than about 25 mm, or less than about 30 mm.

152. The method of claim 147, wherein the cancer comprises a polyp, an adenoma, or a frank cancer.

153. The method of claim 147, wherein the cancer comprises Lynch syndrome cancer, familial adenomatous polyposis, hereditary non-polyposis colon cancer (HNPCC), cholangiocarcinoma, pancreatic ductal adenocarcinoma (PDAC), or a sporadic cancer.

154. The method of claim 147, wherein the cancer is selected from squamous cell carcinoma of anus, low-grade squamous intraepithelial lesions (LSIL) of anus, high-grade squamous intraepithelial lesions (HSIL) of anus, colorectal cancer, colorectal adenocarcinoma, familial adenomatous polyposis, hereditary nonpolyposis colorectal cancer, colorectal polyposis (e.g. Peutz-Jeghers syndrome, juvenile polyposis syndrome, MUTYH-associated polyposis, familial adenomatous polyposis/Gardner's syndrome, Cronkhite-Canada syndrome), carcinoid, pseudomyxoma peritonei, duodenal adenocarcinoma, premalignant adenoma of small bowel, distal bile duct carcinomas, biliary intraepithelial neoplasm (BilIN), BilIN-1, BilIN-2, BilIN-3 or cholangiocarcinoma, pancreatic ductal adenocarcinoma (PDAC), pancreatic intraepithelial neoplasm (PanIN), PanIN-1, PanIN-2, PanIN-3, gastric carcinoma, signet ring cell carcinoma (SRCC), gastric lymphoma (MALT lymphoma), linitis plastic (Brinton's disease), and squamous cell carcinoma of esophagus and adenocarcinoma.

155. A method for treating a cancer in a subject, comprising:
(i) administering to the gastrointestinal tract of the subject the genetically engineered microorganism of any one of claims 92 to 144;
(ii) obtaining a biological sample from the subject; and
(iii) measuring the secretable biomarker in the biological sample to thereby detecting the diseased epithelial cells; and
(iv) administering a treatment if the expression of the secretable biomarker is observed.

156. The method of any one of claims 145-155, wherein the subject is predisposed to develop polyps and/or cancer.

157. The method of claim 156, wherein the subject suffers from a condition selected from Lynch Syndrome, hereditary non-polyposis colon cancer (HNPCC), familial adenomatous polyposis (FAP), Gardner's Syndrome, Turcot's Syndrome, MUTYH-associated polyposis, Peutz-Jeghers syndrome, and juvenile polyposis syndrome and colitis-associated colorectal cancer (CACC).

158. The method of claim 157, wherein the subject has Lynch Syndrome.

159. The method of any one of claims 145 to 158, wherein the treatment is selected from colonoscopy, endoscopy, surgery and administration of a therapeutic agent selected from the group consisting of a chemotherapeutic agent, a cytotoxic agent, an immune checkpoint inhibitor, an immunosuppressive agent, a sulfa drug, a corticosteroid, an antibiotic and a combination of any two or more thereof.
